SEQUENCE LISTING (20 sequences)

SEQ ID NO: 1 - DNA, 2169 bp, Homo sapiens
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcc

SEQ ID NO: 2 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - DNA, 1794 bp, Artificial Sequence
  ACE2 Variant
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 4 - protein, 598 aa, Artificial Sequence
  ACE2 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 5 - DNA, 1794 bp, Artificial Sequence; identical to SEQ ID NO: 3
  ACE2 variant

SEQ ID NO: 6 - protein, 598 aa, Artificial Sequence
  ACE2 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIQYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 7 - DNA, 1794 bp, Artificial Sequence
  ACE2 variant
cagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcataacgagatggggaatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 8 - protein, 598 aa, Artificial Sequence
  ACE2 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 9 - DNA, 576 bp, Homo sapiens
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcatt

SEQ ID NO: 10 - protein, 192 aa, Homo sapiens
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCI

SEQ ID NO: 11 - DNA, 576 bp, Artificial Sequence
  CR1 SCR1-3 variant
caatgcaatgccccagaatggcttccatttgccaggcctaccaacctaactgatgaatttgagtttcccattgggacatatctgaaatatgaatgccgccctggttattacggaagaccgttttctatcatctgcctaaaaaactcagtctggactggtgctaaggacaggtgcagacgtaaatcatgtcgtaatcctccagatcctgtgaatggcatggtgcatgtgatcaaagacatccagttcggatcccaaattaaatattcttgtactaaaggataccgactcattggttcctcgtctgccacatgcatcatctcaggtaatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcatt

SEQ ID NO: 12 - protein, 192 aa, Artificial Sequence
  CR1 SCR1-3 variant
QCNAPEWLPFARPTNLTDEFEFPIGTYLKYECRPGYYGRPFSIICLKNSVWTGAKDRCRRKSCRNPPDPVNGMVHVIKDIQFGSQIKYSCTKGYRLIGSSSATCIISGNTVIWDNETPICDRIPCGLPPTITNGDFISTNRENFHYGSVVTYRCNPGSGGRKVFELVGEPSIYCTSNDDQVGIWSGPAPQCI

SEQ ID NO: 13 - DNA, 567 bp, Homo sapiens
cgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcaga

SEQ ID NO: 14 - protein, 189 aa, Homo sapiens
RSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECR

SEQ ID NO: 15 - DNA, 567 bp, Homo sapiens
agagaaacatgtccatatatacgggatcctttaaatggccaagcagtccctgcaaatgggacttacgagtttggttatcagatgcactttatttgtaatgagggttattacttaattggtgaagaaattctatattgtgaacttaaaggatcagtagcaatttggagcggtaagcccccaatatgtgaaaaggttttgtgtacaccacctccaaaaataaaaaatggaaaacacacctttagtgaagtagaagtatttgagtatcttgatgcagtaacttatagttgtgatcctgcacctggaccagatccattttcacttattggagagagcacgatttattgtggtgacaattcagtgtggagtcgtgctgctccagagtgtaaagtggtcaaatgtcgatttccagtagtcgaaaatggaaaacagatatcaggatttggaaaaaaattttactacaaagcaacagttatgtttgaatgcgataagggtttttacctcgatggcagcgacacaattgtctgtgacagtaacagtacttgggatcccccagttccaaagtgtctt

SEQ ID NO: 16 - protein, 189 aa, Homo sapiens
RETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCL

SEQ ID NO: 17 - DNA, 735 bp, Homo sapiens
gaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaa

SEQ ID NO: 18 - protein, 245 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCE

SEQ ID NO: 19 - DNA, 561 bp, Homo sapiens
aattgtggtcctccacccactttatcatttgctgccccgatggatattacgttgactgagacacgcttcaaaactggaactactctgaaatacacctgcctccctggctacgtcagatcccattcaactcagacgcttacctgtaattctgatggcgaatgggtgtataacaccttctgtatctacaaacgatgcagacacccaggagagttacgtaatgggcaagtagagattaagacagatttatcttttggatcacaaatagaattcagctgttcagaaggatttttcttaattggctcaaccactagtcgttgtgaagtccaagatagaggagttggctggagtcatcctctcccacaatgtgaaattgtcaagtgtaagcctcctccagacatcaggaatggaaggcacagcggtgaagaaaatttctacgcatacggcttttctgtcacctacagctgtgacccccgcttctcactcttgggccatgcctccatttcttgcactgtggagaatgaaacaataggtgtttggagaccaagccctcctacctgtgaa

SEQ ID NO: 20 - protein, 187 aa, Homo sapiens
NCGPPPTLSFAAPMDITLTETRFKTGTTLKYTCLPGYVRSHSTQTLTCNSDGEWVYNTFCIYKRCRHPGELRNGQVEIKTDLSFGSQIEFSCSEGFFLIGSTTSRCEVQDRGVGWSHPLPQCEIVKCKPPPDIRNGRHSGEENFYAYGFSVTYSCDPRFSLLGHASISCTVENETIGVWRPSPPTCE

(19) United States
(12) Patent Application Publication
(10) Pub. No.: US 2021/0292384 A1
(43) Pub. Date: Sep. 23, 2021

(54) Bispecific and trispecific functional molecules of ACE2 and complement pathways and their use

(71) Applicant: Larix Bioscience LLC, Sunnyvale, CA (US)

(72) Inventors: Bo Yu, Sunnyvale, CA (US); James Larrick, Sunnyvale, CA (US)

(73) Assignee: Larix Bioscience LLC, Sunnyvale, CA (US)

(21) Appl. No.: 17/207,567

(22) Filed: Mar. 19, 2021

Related U.S. Application Data
(60) Provisional application No. 62/992,910, filed on Mar. 21, 2020.

(51) Int. Cl. C07K 14/47; A61P 31/14

(52) U.S. Cl. CPC C07K 14/4703; C07K 2319/30; C07K 2319/31; A61P 31/14

(57) ABSTRACT

Disclosed herein are ACE2 fusion proteins that can be used to block infection of cells by SARS-CoV-2. The fusion proteins may have additional functionality such as inhibition of complement. The fusion protein may also include a polypeptide or a moiety that increases serum or blood half-life of the ACE2 fusion protein.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “LRX012_ST25.txt”, a creation date of Mar. 15, 2021, and a size of 48 kilobytes. The Sequence Listing filed via EFS-Web is part of the specification and is incorporated in its entirety by reference herein.

### BACKGROUND

The technical field is biological molecules for treatment of angiotensin-converting enzyme 2 (ACE2)- and complement-related human diseases. More particularly, nucleic acids encoding and polypeptides combining functional ACE2 activity and specific and potent inhibitors of complement pathways are described and can be used to treat viral infection, heart, lung and kidney diseases, hypertension, and inflammation.

The Renin-Angiotensin Aldosterone System (RAAS) plays a central role in the control of cardiovascular and renal functions by maintaining homeostasis of blood pressure and electrolyte balance. Abnormal activation of the RAAS is associated with the pathogenesis of cardiovascular and renal diseases such as hypertension, myocardial infarction and heart failure (Jai, 2016). The protease renin cleaves angiotensinogen into the inactive decameric peptide angiotensin I (Ang I). The angiotensin-converting enzyme (ACE) then cleaves the Ang 1 into the active octomer angiotensin II (Ang II), which promotes vascular smooth muscle vasoconstriction and renal tubule sodium reabsorption. The angiotensin-converting enzyme 2 (ACE2) is a membrane carboxypeptidase expressed primarily in lung, kidney, heart, and endothelial cells, and cleaves various peptide substrates. ACE2 hydrolyses Ang I to generate Ang 1-9, and Ang II to generate Ang 1-7. Ang 1-7 exhibits vasodilating effects and antagonizes many of the Ang II-mediated effects. Overall, ACE2 functions as a RAAS counter-regulatory enzyme by decreasing local Ang II concentrations (Imai, 2010).

ACE2 has been demonstrated to be protective in several acute respiratory distress syndrome (ARDS) models, and models of pulmonary fibrosis and pulmonary hypertension. Recombinant human ACE2 (rhACE2) has been studied in a pilot phase II clinical trial for acute lung injury (ALI). Administration of a broad range of doses of rhACE2 was safe without causing significant hemodynamic changes. Twice-daily infusion resulted in a rapid decrease in plasma Ang II levels and an increase in Ang 1-7 and Ang 1-5 levels, as well as a trend toward a decrease in plasma IL-6 concentrations (Khan, 2017). In addition, rhACE2 provided beneficial effects against Ang II-induced heart failure with preserved ejection fraction (HFpEF) and pressure-overload-induced HF with reduced ejection fraction in murine models of HF (Patel, 2016). Although rhACE2 has exhibited potential therapeutic applications in pulmonary and cardiovascular diseases, its relative short half-life has limited its clinical development.

ACE2 receptors have also been shown to be the human receptor for some coronaviruses, including SARS-CoV (Kuba, 2005) and SARS-CoV-2 (Hoffmann, 2020). Viral entry for SARS-CoV is mediated by the spike glycoprotein (S protein) expressed on the virion surface, which facilitates receptor recognition and membrane fusion (Gallagher, 2001; Belouzard, 2012). During viral infection, the trimeric S protein is cleaved into S1 and S2 subunits, and S1 subunits are released in the transition to the post-fusion conformation (Song, 2018). S1 contains the receptor binding domain (RBD), which directly binds to the peptidase domain (PD) of ACE2, while S2 is responsible for membrane fusion. When S1 binds to the host receptor ACE2, another cleavage site on S2 is exposed and is cleaved by host proteases, a process that is critical for viral infection (Li, 2016). Soluble ACE2 has been shown to bind the S1 subunit and block the viral entry into the human cells. A number of studies have identified that this viral entry point is the same for SARS-CoV-2, the virus that causes COVID-19 which is an emerging global human health threat with a more than 2% case fatality rate (Zhang, 2020a; Zhou, 2020; Zhu, 2020). In addition, binding of the SARS-CoV spike protein down-regulated ACE2 expression in lung resulting in elevated Ang II that exacerbated the ARDS symptoms (Kuba, 2005). A recombinant ACE2 (rACE2) protein could block viral entry into the cells by binding to the spike protein and reduce the ARDS symptoms by cleaving Ang II. Thus rACE2 may have an important place in protecting ARDS patients as well as a potential therapeutic approach for the management of emerging lung diseases such as SARS, avian influenza A infections, Covid-19, etc. (Zou, 2014).

The complement system is a critical part of host defense to many bacterial, viral, and fungal infections. It works alongside pattern recognition receptors (PRR) to stimulate host defense systems in advance of activation of the adaptive immune response. Activation of the complement leads to a series of protease activation cascade triggering release of cytokines and amplification of the activation cascade. Delicate balance between defense against pathogen and avoidance of excess inflammation has to be achieved for the complement system. Many inflammatory, autoimmune, neurodegenerative and infectious diseases have been shown to be associated with excessive complement activity.

The complement system can be activated through three different pathways: the classical pathway, the alternative pathway, and the lectin pathway (Wagner, 2010). All three pathways converge of the critical protease complexes of C3-convertase and C5-convertase that cleave complement components C3 and C5, respectively. Activation of the complement system leads to formation of the cell-killing membrane attack complex (MAC), inflammation caused by anaphylatoxins C3a and C5a, and opsonization of pathogens. The MAC is essential for eliminating invading pathogens and damaged, necrotic, and apoptotic cells.

The classical pathway is initiated by binding of C1q to antibodies IgM or IgG leading to activation of the C1 complex that cleaves complement components C2 and C4 to form the classical pathway C3-convertase and then the C5-convertase.

The lectin pathway is initiated by binding of mannose-binding lectin (MBL) to mannose residues on the pathogen surface, which leads to forming of the same C3-convertase and C5-convertase as in the classical pathway.

Unlike the classical and the lectin pathways that are specific immune responses requiring antigens, the alternative pathway is a non-specific immune response that is continuously active at a low level. Spontaneous hydrolysis of C3 leads to formation of the C3-convertase of the alternative pathway and then the C5-convertase of the alternative pathway. The C5-convertases from all three pathways can cleave C5 to C5a and C5b which recruits and assembles C6, C7, C7, C8 and multiple C9 molecules to assemble the MAC. This creates a hole or pore in the membrane that can kill or damage the pathogen or cell.

The complement system is tightly regulated by decay accelerating activity (DAA) and cofactor activity (CA). DAA refers to the ability to promote dissociation of the C3-convertase or C5-convertase. CA refers to the ability of facilitating Factor I to inactivate the C3-convertase or C5-convertase. There are a number of complement regulators that exhibit DAA or CA toward the classic or alternative pathways. Decay-accelerating factor (DAF) accelerates the dissociation of both classical and alternative C3-convertases. Membrane cofactor protein (MCP) is a cofactor for Factor I-mediated cleavage of C3b to iC3b. Factor H and C4 binding protein (C4BP) have DAA towards the alternative and classical pathway C3-convertases, respectively, and CAs for the cleavage of C3b and C4b. Human complement Receptor type 1 (CR1) is the only complement regulator that has DAA for the both classical and alternative C3-convertases and C5-convertases and CA for C3b and C4b. In addition to regulators of DAA and CA, the other complement regulators are complement-associated protease inhibitor C1-INH which inhibits cleavage of C4 and formation of the classical C3-convertase and Protectin (CD59) which inhibits MAC directly and prevents cell lysis.

Human CR1 is a large glycoprotein (˜200 kD) consisting of 30 repeating homologous short consensus repeats (SCR, 60-70aa) followed by transmembrane and cytoplasmic domains. Most of the DAA but not CA activities map to the first 3 SCRs (SCR1-3) (Krych-Goldberg, 1999). The other complement regulatory proteins DAF, MCP, Factor H, and C4BP also contain a number of SCRs where the active sites for complement inhibitions have been mapped by deletion analysis to a few SCRs (Makrides, 1998). SCR2-4 in DAF binds C3b and C4b and has DAA for the C3-convertases. SCR2-4 in MCP binds C3b and C4b and has CA for C3b and C4b. SCR1-4 in Factor H binds C3b and has CA for C3b and DAA for the alternative C3-convertase. SCR1-3 in C4BP a subunit binds C4b and has CA for C4b and DAA for the classical C3-convertase.

Human Complement Receptor type 1 (CR1) has generated interest for therapeutic application (Krych-Goldberg, 2001). Soluble CR1 extracellular domain was shown to inhibit the complement system in vivo (Mollnes, 2006), and safely and effectively reduced tissue damage from myocardial infarction in human clinical trials (Perry, 1998), adult respiratory distress syndrome (Zimmerman, 2000), and lung transplantation (Zamora, 1999). A number of other complement regulator proteins, e.g. MCP, DAF, and Protectin, effectively inhibit complement in vitro and in various animal models (Wagner, 2010). Several engineered fusion proteins have been constructed with complement regulators to improve efficacy. MCP and DAF were fused to generate Complement Activity Blocker 2 (CAB2). Factor H and the C3 fragment-binding domain of the Complement Receptor type 2 (CR2) were fused to generate TT30. A humanized antibody fragment directed against Factor B and the C3d-binding domain of CR2 was fused to generate TA106.

Monoclonal antibodies against complement proteins have also been used as therapeutic agents (Ehrnthaller, 2011). Anti-05 Eculizumab was approved to treat paroxysmal nocturnal hemoglobinuria (PNH) in 2007. Antibodies against C5a (TNX-558), Factor D (TNX-234), Factor P, and C3b, an aptamer inhibitor of human C5 (ARC1905), and a 13-amino acid cyclic peptide (Compastatin) against C3 have been developed and evaluated in various disease models and human clinical trials (Ricklin, 2016; Mastellos, 2017; Ricklin, 2017; Zipfel, 2019).

Disorders with known or suspected complement involvement cover an exceptionally broad range, including tissue-specific, systemic, acute and chronic disorders of the inflammatory, autoimmune, age-related, biomaterial-induced and neurodegenerative spectrum. An overwhelming number of activating triggers, such as pattern-associated molecular patterns (PAMPs) in the case of sepsis or damage-associated molecular patterns (DAMPs) in trauma, can lead to systemic inflammatory response syndrome (SIRS), in which the severe and sudden reaction of complement and other defense pathways causes homeostatic imbalance, hyper-acute inflammation and tissue damage that can lead to organ dysfunction and death. In the case of complement involvement, too much of a protective response can lead to an adverse outcome. In transplant-induced and bio-material-induced inflammation, complement recognizes non-self surfaces that are exposed to blood or tissue fluid and invokes an appropriate but unwanted response. The subsequent adverse reactions might have a negative impact on the quality of life of the patient and on the lifetime and function of the foreign component and can in extremis lead to rejection of the material, cell or organ.

In addition to acute inflammatory conditions, complement drives several chronic disorders, such as PNH, atypical hemolytic uremic syndrome (aHUS) and age-related macular degeneration (AMD). The majority of these disorders are at least partially mediated by unbalanced complement activation due to alterations in complement genes and proteins, including mutations, polymorphisms, deletions and deficiencies. Polymorphisms can lead to gain-of-function alterations of complement activators or loss-of-function alterations of complement regulators and may impair the self-recognition capabilities of soluble complement regulators such as factor H. The distinct combination of complement alterations in an individual, sometimes referred to as the complotype, often determines the fitness of his or her complement system and his or her susceptibility to certain diseases.

The normally beneficial actions of complement aimed at removing immune complexes, apoptotic cells and debris can lead to adverse reactions when debris or plaque can no longer be removed, resulting in constant complement activation that contributes to an inflammatory microenvironment. Prominent examples of this principle are age-related disorders such as Alzheimer disease, atherosclerosis and AMD. In addition, insufficient clearance of apoptotic cells and/or immune complexes owing to deficiencies in early complement components is considered to be a key contributor to autoimmune diseases such as systemic lupus erythematosus (SLE).

The pronounced susceptibility of the kidney to complement-mediated injury has been largely attributed to its unique anatomical and functional features that seem to be conducive to complement activation. Similar to the eyes, which are also particularly susceptible to disorders driven by imbalanced complement activation, the kidneys are characterized by a prominent, compartment-dividing membrane, the glomerular basement membrane, that lacks complement regulators and may be prone to attack upon damage of the covering cell layer. Complement activation contributes to many kidney diseases among them: aHUS, C3 glomerulopathies, which include dense deposit disease and C3 glomerulonephritis, complications of hemodialysis and kidney transplantation, diabetic nephropathy, IgA nephropathy, lupus nephritis and anti-neutrophil cytoplasmic antibody (ANCA)-associated vasculitis (AAV).

Blockage of the complement system has been shown to be attenuate the pathogenesis of SARS-CoV (Gralinski, 2018) and MERS-CoV (Jiang, 2018). Complement activation measured by C3 cleavage was markedly elevated in the lungs of C57BL/6J mice infected with SARS-CoV MA15. Mice deficient in C3 (C3−/−) were protected from SARS-CoV-induced weight loss and exhibited less pathology, improved respiratory function, and lower levels of inflammatory cytokines/chemokines in the lung and periphery. The kinetics and magnitude of virus replication in C3−/− and wild-type mice were the same, showing that complement does not play a role in controlling viral replication. Complement deposition in the lungs of SARS-CoV-infected mice suggests that complement activation results in immune-mediated damage to the lung. Additionally, serum activation indicates that complement-mediated systemic inflammation may drive the pathogenic response to SARS-CoV infection. Furthermore, the complement system has well-described roles in other pulmonary diseases (Sarma, 2006), especially after influenza virus and respiratory syncytial virus infection (Chang, 2010; Thielens, 2002; Bera, 2011). Importantly, MERS-CoV and H5N1 influenza virus-induced acute lung injury and pulmonary inflammation are reduced in mice that are treated with either a C3a receptor (C3aR) antagonist or antibodies to C5a (Jiang, 2018; Sun, 2013). Together, these results indicate a critical role for complement in the pathogenesis of SARS-CoV and suggest that inhibition of the complement pathway could effectively augment anti-viral therapeutics in coronavirus-mediated disease.

Preliminary pathology studies of COVID-19 patients showed diffuse alveolar damage with edema, hyaline membranes, and inflammation, followed by type II pneumocyte hyperplasia, features characteristic of typical ARDS (Xu, 2020; Zhang, 2020b). Proinflammatory cytokines and chemokines including TNFα, IL-1β, IL-6, G-CSF, IP-10, MCP-1, and MIP-1-α were significantly elevated in COVID-19 patients (Huang, 2020; Liu, 2020). Clinical observation of severe COVID-19 patients included elevated LDH, d-dimer, and bilirubin; decreased platelets; mild anemia; renal and cardiac injury; and reportedly diffuse thrombotic microangiopathy (TMA), consistent with abnormal coagulation and excessive complement activation (Zhou, 2020b). Severe COVID-19 may define a type of catastrophic microvascular injury syndrome mediated by activation of complement pathways and an associated procoagulant state (Campbell, 2020; Magro, 2020).

The N proteins of SARS-CoV-1, MERS-CoV, and SARS-CoV-2 were found to bind to MASP-2, the key serine protease in the lectin pathway (LP) of complement activation, resulting in aberrant complement activation and aggravated inflammatory lung injury (Gao, 2020). Clinical evidence of the over-activation of complement LP pathway was identified in COVID-19 patients. Significantly increased serum C5a level was also observed in COVID-19 patients, particularly in severe cases. These results collectively indicated that complement pathways are aggressively activated in the lungs of COVID-19 patients, which may be attributed to abundant interactions between SARS-CoV-2 N protein and MASP-2 (Gao, 2020). Complement suppression may represent an attractive common therapeutic approach for pneumonia induced by these highly pathogenic coronaviruses.

Since the spike protein is critical for SARS-CoV-2 viral entry, it has been targeted for vaccine development and therapeutic antibody interventions. The current suite of antibody therapeutics and vaccines was designed with a spike protein based on strains circulating during the early phases of the pandemic in 2020. More recently, variants with enhanced transmissibility have emerged in the United Kingdom (B.1.1.7), South Africa (B.1.351), Brazil (B.1.1.248) and elsewhere with multiple substitutions in the spike protein. Several studies indicated that neutralization by some antibodies and immune sera are diminished against variants expressing mutations in the spike gene (Chen, 2021; Wang, 2021). Decoy receptors based on soluble ACE2 are expected to maintain the neutralization activity against the emerging SARS-CoV-2 variants as long as they continue to utilize ACE2 as the cell receptor for infection.

### SUMMARY

The present disclosure describes a series of fusion proteins that contain human ACE2 and a specific inhibitor to the complement pathway. These fusion proteins contain domains of human proteins and are of human sequences, and thus are expected to be low or non-immunogenic and can be used as therapeutics in human to treat ACE2 and complement-related diseases. In addition, the described fusion proteins are comprised of domains that facilitate long serum half-life.

The disclosure also describes human ACE2 variants that have enhanced affinity for SARS-CoV-2 spike protein. The human ACE2 variants include those described in Table 1. The variants in Table 1 can be used in the polypeptides of SEQ ID NO: 2, 4, 6, or 8. The enhanced affinity ACE2 variants can be used to treat subjects with SARS-CoV-2. Administration of these enhanced affinity ACE2 variants can compete for binding to ACE2 in the subject and so block SARS-CoV-2 from infecting cells in the subject.

The polypeptides described herein can ameliorate activation of two major pathways that contribute to the pathogenesis of many diseases; complement and the renin-angiotensin-aldosterone system. Polypeptides described herein can reduce the activation of these pathways. The polypeptides described herein can be used to treat patients suffering from various chronic fibrotic diseases, among them idiopathic pulmonary fibrosis, pulmonary artery hypertension, congestive heart failure, hepatic fibrotic disease and NASH, chronic kidney disease of diverse origin and progressive systemic sclerosis. The polypeptides described herein can also be used to treat patients suffering from acute respiratory distress syndrome (ARDS) that accompanies serious coronavirus infections, as well as patients with ARDS of diverse etiology, e.g. sepsis, multiple organ failure, pulmonary emboli, etc.

Nucleic acids encoding the polypeptides described herein can be used to make these polypeptides in vitro or in vivo.

### BRIEF DESCRIPTION OF THE DRAWING

FIG. 1 is schematic drawings of the fusion proteins. Domains ACE2 and ACE2V are the soluble extracellular domains of the human ACE2 and its peptidase inactive variant (R273K or N374N378). CID is a complement inhibitor. HSA is the human albumin. Fc is the human IgG4 Fc region. TrD is a trimeric forming domain.

FIG. 2 is a schematic drawing of additional fusion proteins.

FIG. 3 is a depiction of an SDS-PAGE for purified ACE2 fusion proteins.

FIG. 4 panels A and B show binding of the ACE2 fusion proteins to SARS-CoV-2 spike protein.

FIG. 5 panels A-E show binding of ACE2 variant fusion proteins to SARS-CoV-2 spike protein.

FIG. 6A shows inhibition of the classic complement pathway by an ACE2 fusion protein.

FIG. 6B shows inhibition of the alternative complement pathway by an ACE2 fusion protein.

FIG. 7 panels A-C show in vitro blocking of pseudotyped SARS-CoV-2 by ACE2 fusion proteins.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 μg, it is intended that the concentration be understood to be at least approximately or about 10 μg.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, the terms “amino acid substitution” or “amino acid difference” are defined to mean a change in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. In most instances herein, the specific amino acid substitution or amino acid residue difference at a position is indicated as “XnY” where “Xn” specifies the corresponding position as described above, and “Y” is the single letter identifier of the amino acid found in the engineered polypeptide (i.e., the different residue than in the reference polypeptide). More than one amino acid can appear at a specified residue position, the alternative amino acids can be listed in the form XnY/Z, where Y and Z represent alternate amino acid residues. In some instances, the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. Furthermore, in some instances, a polypeptide of the present disclosure can include one or more amino acid residue differences relative to a reference sequence, which is indicated by a list of the specified positions where changes are made relative to the reference sequence.

As used herein, an “antibody” is defined to be a protein functionally defined as a ligand-binding protein and structurally defined as comprising an amino acid sequence that is recognized by one of skill as being derived from the variable region of an immunoglobulin. An antibody can consist of one or more polypeptides substantially encoded by immunoglobulin genes, fragments of immunoglobulin genes, hybrid immunoglobulin genes (made by combining the genetic information from different animals), or synthetic immunoglobulin genes. The recognized, native, immunoglobulin genes include the kappa, lambda, alpha, gamma, delta, epsilon and mu constant region genes, as well as myriad immunoglobulin variable region genes and multiple D-segments and J-segments. Light chains are classified as either kappa or lambda. Heavy chains are classified as gamma, mu, alpha, delta, or epsilon, which in turn define the immunoglobulin classes, IgG, IgM, IgA, IgD and IgE, respectively. Antibodies exist as intact immunoglobulins, as a number of well characterized fragments produced by digestion with various peptidases, or as a variety of fragments made by recombinant DNA technology. Antibodies can derive from many different species (e.g., rabbit, sheep, camel, human, or rodent, such as mouse or rat), or can be synthetic. Antibodies can be chimeric, humanized, or humaneered. Antibodies can be monoclonal or polyclonal, multiple or single chained, fragments or intact immunoglobulins.

As used herein, an “antibody fragment” is defined to be at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific antibodies formed from antibody fragments. The term “scFv” is defined to be a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “codon optimized” is defined to mean changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. The polynucleotides encoding the polypeptides described herein may be codon optimized for optimal production from the host organism selected for expression.

As used herein, the terms “consensus sequence” and “canonical sequence” are defined to mean an archetypical amino acid sequence against which all variants of a particular protein or sequence of interest are compared. The terms also refer to a sequence that sets forth the nucleotides that are most often present in a DNA sequence of interest. For each position of a gene, the consensus sequence gives the amino acid that is most abundant in that position in a multiple sequence alignment (MSA).

As used herein, the terms “conservative amino acid substitution” or “conservative amino acid difference” are defined to mean a change in the amino acid at a residue position to a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, and isoleucine; an amino acid with hydroxyl side chain is substituted with another amino acid with a hydroxyl side chain, e.g., serine and threonine; an amino acid having aromatic side chains is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively. Exemplary conservative substitutions are provided in Table 1 below.

TABLE 1

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | None |

As used herein, the term “control sequence” is defined to include all components, which are necessary or advantageous for the expression of a polynucleotide and/or polypeptide of the present disclosure. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and where appropriate, translational stop signals. The control sequences may be provided with linkers for the purpose of introducing specific restriction sites facilitating ligation of the control sequences with the coding region of the nucleic acid sequence encoding a polypeptide.

As used herein, an “effective amount” or “therapeutically effective amount” are used interchangeably, and defined to be an amount of a compound, formulation, material, or composition, as described herein effective to achieve a particular biological result.

As used herein, “heterologous” is defined to mean the nucleic acid and/or polypeptide is not homologous to the host cell. Alternatively, “heterologous” means that portions of a nucleic acid or polypeptide that are joined together to make a combination where the portions are from different species, and the combination is not found in nature.

As used herein, the term “homologous genes” is defined to mean a pair of genes which correspond to each other and which are identical or similar to each other. The term encompasses genes that are separated by speciation (i.e., the development of new species) (e.g., orthologous genes), as well as genes that have been separated by genetic duplication (e.g., paralogous genes).

As used herein, the term “isolated polypeptide” is defined to mean a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis).

As used herein, the terms “non-conservative substitution” or “non-conservative amino acid difference” are defined to mean a change in the amino acid at a residue position to a different residue with significantly differing side chain properties. Non-conservative substitutions may use amino acids between, rather than within, the defined groups and affects (a) the structure of the peptide backbone in the area of the substitution (e.g., proline for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, the terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included.

As used herein, the term “operably linked” is defined to mean a configuration in which a control sequence is appropriately placed (i.e., in a functional relationship) at a position relative to a polynucleotide of interest such that the control sequence directs or regulates the expression of the polynucleotide and/or polypeptide of interest.

As used herein, the terms “ortholog” and “orthologous genes” are defined to mean genes in different species that have evolved from a common ancestral gene (i.e., a homologous gene) by speciation. Typically, orthologs retain the same function during the course of evolution. Identification of orthologs finds use in the reliable prediction of gene function in newly sequenced genomes.

As used herein, the terms “paralog” and “paralogous genes” are defined to mean genes that are related by duplication within a genome. Generally, paralogs tend to evolve into new functions, even though some functions are often related to the original one.

As used herein, the terms “polynucleotide” or “nucleic acid′ are used interchangeably and are defined to mean two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. Preferably, such modified or synthetic nucleobases will be encoding nucleobases.

As used herein, the term “promoter sequence” is defined to mean a nucleic acid sequence that is recognized by a host cell for expression of a polynucleotide of interest, such as a coding sequence or gene. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

As used herein, the terms “protein”, “polypeptide,” and “peptide” are used interchangeably and are defined to mean a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids. The standard single or three letter abbreviations can be used for the genetically encoded amino acids (see, e.g., IUPAC-IUB Joint Commission on Biochemical Nomenclature, “Nomenclature and Symbolism for Amino Acids and Peptides,” Eur. J. Biochem. 138:9-37, 1984).

As used herein, the terms “recombinant” or “engineered” or “non-naturally occurring” are used interchangeably and are defined to mean modified polypeptides or nucleic acids which polypeptides or nucleic acids are modified in a manner that would not otherwise exist in nature, or is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

As used herein, the term “stringent hybridization conditions” is defined to mean hybridizing in 50% formamide at 5×SSC at a temperature of 42° C. and washing the filters in 0.2×SSC at 60° C. (1×SSC is 0.15M NaCl, 0.015M sodium citrate.) Stringent hybridization conditions also encompasses low ionic strength and high temperature for washing, for example 0.015 M sodium chloride/0.0015 M sodium citrate/0.1% sodium dodecyl sulfate at 50° C.; hybridization with a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

As used herein, the term “substantially pure polypeptide” is defined to mean a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure composition will comprise about 60% or more, about 70% or more, about 80% or more, about 90% or more, about 95% or more, and about 98% or more of all macromolecular species by mole or % weight present in the composition. The object species can be purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. An isolated engineered polypeptide can be a substantially pure polypeptide composition.

As used herein, the terms “wild-type” is defined to mean the form found predominantly in nature. For example, a wild-type polypeptide or polynucleotide sequence is a sequence predominantly present in an organism that can be isolated from a source in nature and which has not been intentionally modified by human manipulation.

### ACE2 Molecules

Recombinant ACE2 provides therapeutic benefits in a number of infection diseases, cardiovascular, renal, and pulmonary diseases, in which inhibition of the complement system is often protective. Described herein are a series of fusion proteins of a human ACE2 domain and complement inhibitors, which take advantage of both functional moieties to provide maximal therapeutic benefits in the related diseases. Particularly in the case of the coronavirus SARS-CoV and SARS-CoV-2 infection, ACE2 is used as cell surface receptor by the viral spike protein for cell entry. Thus, a soluble ACE2 fusion protein could be used as a decoy for the viral spike protein to block the cell entry.

The ACE2 domain could be the full length ACE2 extracellular domain (SEQ ID NO: 1-2), or a deletion variant (SEQ ID NO: 3-4). It could maintain the peptidase activity or be enzymatically inactive (SEQ ID NO: 5-8). It could be engineered to enhance peptidase activity or binding affinity to the coronaviral spike proteins. It could be any variants with different activities.

The complement inhibitors described in the present invention could be any proteins or protein fragments that inhibit the complement activation, such as domains of human CR1, DAF, MCP, Factor H, C4BP, Protectin, C1-INH, and their counterparts in other primates. In particular, they could be the human CR1 SCR1-3 (SEQ ID NO: 9-10), its variant containing mutations of N29K/S37Y/G79D/D109N (SEQ ID NO: 11-12), DAF SCR2-4 (SEQ ID NO: 13-14), MCP SCR2-4 (SEQ ID NO: 15-16), Factor H SCR1-4 (SEQ ID NO: 17-18), or C4BPA SCR1-3 (SEQ ID NO: 19-20). Among them, the Factor H SCR1-4 is the only CID that specifically targets the alternative pathway but not the classical pathway. These complement inhibitors could be mutagenized to enhance the inhibitory activities, and all such improvements fall within the scope of the present invention. The complement inhibitors could also be any peptide inhibitors or oligonucleotide inhibitors against Factor B, Factor D, Factor P, C3, or C5. They could also be any full-length or fragments of antibodies, or the antibody variable regions (VH or VK), or the scFv antibodies derived from antibodies against Factor B, Factor D, Factor P, C3, or C5.

In addition, these fusion proteins of ACE2 and complement inhibitor are conjugated with a third moiety to prolong in vivo half-life. The third moiety could be the human serum albumin (HSA) which is monomeric, the immunoglobulin IgG4 Fc or its stabilized and FcγR non-binding variant which is dimeric, a human collagen COL18A1 trimeric domain, or any other domains that prolong the in vivo half-life. The Fc domain could be from any immunoglobulin isotypes, subclasses, and allotypes. These fusion proteins could also be PEGylated or conjugated with polymers to increase half-life in vivo; or chemically cross-linked to antibodies, fragments of antibodies, Fc regions, HSA, or other human proteins to increase half-life in vivo; or formulated in any long-term sustained releasing format (e.g. lipid nanoparticles, etc.) to prolong the ACE2 and anti-complement activities in vivo.

The ACE2 polypeptides are include ACE2 variants that have enhanced affinity for the spike protein of SARS-CoV-2. Examples of these ACE2 variants are found in Table 1 which amino acid changes can be placed into the ACE2 polypeptides of SEQ ID NO: 2, 4, 6, and/or 8. The ACE2 variants can include SEQ ID NO: 2, 4, 6, and/or 8 with one or more of the following amino acid substitutions: K26E, K26R, T27R, F28W, D30E, K31E, Y41N, Q42E, L45E, L79W, Y83F, G326E, N330K, N330Q, N330Y, G352Y, and/or K353H. The ACE2 variants can include SEQ ID NO: 2, 4, 6, and/or 8 with one or more of the following double amino acid substitutions: K26R/L79W, T27R/L79W, F28W/L79W, D30E/L79W, and/or Q42E/L79W. The ACE2 variants can include SEQ ID NO: 2, 4, 6, and/or 8 with one or more of the following triple amino acid substitutions: K26R/D30E/L79W, D30E/L79W/N330Q, D30E/L79W/N330Y, and/or D30E/Q42K/L79W. The ACE2 variants can include SEQ ID NO: 2, 4, 6, and/or 8 with one or more of the following quadruple amino acid substitutions: K26R/F28W/D30E/L79W, D30E/Q42K/L79W/N330Q, D30E/Q42K/L79W/N330Y, and/or K31F/N33D/H34S/E35Q. The ACE2 variants can include SEQ ID NO: 2, 4, 6, and/or 8 with one or more of the following quintuple amino acid substitutions: K26R/T27R/F28W/D30E/L79W, T27R/D30E/Q42K/L79W/N330Y, T27Y/D30E/Q42K/L79W/N330Y, and/or D30E/H34V/Q42K/L79W/N330Y. The ACE2 variants can include SEQ ID NO: 2, 4, 6, and/or 8 with the following septuple amino acid substitution: D30E/K31F/H34I/E35Q/Q42K/L79W/N330Y.

The ACE2 variants can include SEQ ID NO: 2 with one or more of the following amino acid substitutions: K26E, K26R, T27R, F28W, D30E, K31E, Y41N, Q42E, L45E, L79W, Y83F, G326E, N330K, N330Q, N330Y, G352Y, and/or K353H. The ACE2 variants can include SEQ ID NO: 2 with one or more of the following double amino acid substitutions: K26R/L79W, T27R/L79W, F28W/L79W, D30E/L79W, and/or Q42E/L79W. The ACE2 variants can include SEQ ID NO: 2 with one or more of the following triple amino acid substitutions: K26R/D30E/L79W, D30E/L79W/N330Q, D30E/L79W/N330Y, and/or D30E/Q42K/L79W. The ACE2 variants can include SEQ ID NO: 2 with one or more of the following quadruple amino acid substitutions: K26R/F28W/D30E/L79W, D30E/Q42K/L79W/N330Q, D30E/Q42K/L79W/N330Y, and/or K31F/N33D/H34S/E35Q. The ACE2 variants can include SEQ ID NO: 2 with one or more of the following quintuple amino acid substitutions: K26R/T27R/F28W/D30E/L79W, T27R/D30E/Q42K/L79W/N330Y, T27Y/D30E/Q42K/L79W/N330Y, and/or D30E/H34V/Q42K/L79W/N330Y. The ACE2 variants can include SEQ ID NO: 2 with the following septuple amino acid substitution: D30E/K31F/H34I/E35Q/Q42K/L79W/N330Y. The ACE2 variants can include SEQ ID NO: 4 with one or more of the following amino acid substitutions: K26E, K26R, T27R, F28W, D30E, K31E, Y41N, Q42E, L45E, L79W, Y83F, G326E, N330K, N330Q, N330Y, G352Y, and/or K353H. The ACE2 variants can include SEQ ID NO: 4 with one or more of the following double amino acid substitutions: K26R/L79W, T27R/L79W, F28W/L79W, D30E/L79W, and/or Q42E/L79W. The ACE2 variants can include SEQ ID NO: 4 with one or more of the following triple amino acid substitutions: K26R/D30E/L79W, D30E/L79W/N330Q, D30E/L79W/N330Y, and/or D30E/Q42K/L79W. The ACE2 variants can include SEQ ID NO: 4 with one or more of the following quadruple amino acid substitutions: K26R/F28W/D30E/L79W, D30E/Q42K/L79W/N330Q, D30E/Q42K/L79W/N330Y, and/or K31F/N33D/H34S/E35Q. The ACE2 variants can include SEQ ID NO: 4 with one or more of the following quintuple amino acid substitutions: K26R/T27R/F28W/D30E/L79W, T27R/D30E/Q42K/L79W/N330Y, T27Y/D30E/Q42K/L79W/N330Y, and/or D30E/H34V/Q42K/L79W/N330Y. The ACE2 variants can include SEQ ID NO: 4 with the following septuple amino acid substitution: D30E/K31F/H34I/E35Q/Q42K/L79W/N330Y. The ACE2 variants can include SEQ ID NO: 6 with one or more of the following amino acid substitutions: K26E, K26R, T27R, F28W, D30E, K31E, Y41N, Q42E, L45E, L79W, Y83F, G326E, N330K, N330Q, N330Y, G352Y, and/or K353H. The ACE2 variants can include SEQ ID NO: 6 with one or more of the following double amino acid substitutions: K26R/L79W, T27R/L79W, F28W/L79W, D30E/L79W, and/or Q42E/L79W. The ACE2 variants can include SEQ ID NO: 6 with one or more of the following triple amino acid substitutions: K26R/D30E/L79W, D30E/L79W/N330Q, D30E/L79W/N330Y, and/or D30E/Q42K/L79W. The ACE2 variants can include SEQ ID NO: 6 with one or more of the following quadruple amino acid substitutions: K26R/F28W/D30E/L79W, D30E/Q42K/L79W/N330Q, D30E/Q42K/L79W/N330Y, and/or K31F/N33D/H34S/E35Q. The ACE2 variants can include SEQ ID NO: 6 with one or more of the following quintuple amino acid substitutions: K26R/T27R/F28W/D30E/L79W, T27R/D30E/Q42K/L79W/N330Y, T27Y/D30E/Q42K/L79W/N330Y, and/or D30E/H34V/Q42K/L79W/N330Y. The ACE2 variants can include SEQ ID NO: 6 with the following septuple amino acid substitution: D30E/K31F/H34I/E35Q/Q42K/L79W/N330Y. The ACE2 variants can include SEQ ID NO: 8 with one or more of the following amino acid substitutions: K26E, K26R, T27R, F28W, D30E, K31E, Y41N, Q42E, L45E, L79W, Y83F, G326E, N330K, N330Q, N330Y, G352Y, and/or K353H. The ACE2 variants can include SEQ ID NO: 8 with one or more of the following double amino acid substitutions: K26R/L79W, T27R/L79W, F28W/L79W, D30E/L79W, and/or Q42E/L79W. The ACE2 variants can include SEQ ID NO: 8 with one or more of the following triple amino acid substitutions: K26R/D30E/L79W, D30E/L79W/N330Q, D30E/L79W/N330Y, and/or D30E/Q42K/L79W. The ACE2 variants can include SEQ ID NO: 8 with one or more of the following quadruple amino acid substitutions: K26R/F28W/D30E/L79W, D30E/Q42K/L79W/N330Q, D30E/Q42K/L79W/N330Y, and/or K31F/N33D/H34S/E35Q. The ACE2 variants can include SEQ ID NO: 8 with one or more of the following quintuple amino acid substitutions: K26R/T27R/F28W/D30E/L79W, T27R/D30E/Q42K/L79W/N330Y, T27Y/D30E/Q42K/L79W/N330Y, and/or D30E/H34V/Q42K/L79W/N330Y. The ACE2 variants can include SEQ ID NO: 8 with the following septuple amino acid substitution: D30E/K31F/H34I/E35Q/Q42K/L79W/N330Y.

These ACE2 variants can be used to treat subjects infected with SARS-CoV-2. These ACE2 variants can also be used prophylactically to protect a subject from infection by SARS-CoV-2. These variants can compete with the ACE2 on the subjects cells for binding to the SARS-CoV-2 spike protein, and so, the ACE2 variants can block SARS-CoV-2 from infecting cells of the subject.

Example 1 describes construction and expression of the ACE2 fusion proteins. The sequence of the human ACE2 extracellular domain is shown in SEQ ID NO: 1-4, whereas that of the enzymatically inactive ACE2 variants are shown in SEQ ID NO: 5-8. Example 2 describes construction and expression of the ACE2 and the complement inhibitor fusion proteins. The sequence of the human CR1 SCR1-3_N29K/S37Y/G79D/D109N is shown in SEQ ID NO: 11-12. It is fused to the C terminus of the ACE2 fusion proteins described in Example 1. The binding activities of the fusion proteins to SARS-COV2 spike protein S1 are assayed in a direct ELISA binding experiment described in Example 3.

The inhibitory activities of the classical complement pathway are characterized in Example 4, whereas that of the alternative complement pathway are characterized in Example 5. The peptidase activities of the fusion proteins are assessed in Example 6 utilizing a fluorogenic peptide substrate. Blocking of the viral entry by the fusion proteins is assessed in Example 7.

The in vivo pharmacokinetic profiles of the fusion proteins are determined in Example 8. The protective activities of the fusion proteins in vivo are characterized in Examples 9-11.

### Polynucleotides and Expression Vectors

In another aspect, polynucleotides can encode any of the engineered ACE2 molecules described herein. Exemplary ACE2 nucleotides are found at SEQ ID NO: 1, 3, 5, and 7 (the corresponding amino acid sequences for the ACE2 are found at SEQ ID NO: 2, 4, 6, and 8). SEQ ID NO: 1 encodes the human, wild-type ACE2 extracellular domain. SEQ ID NO: 3, 5 and 7 encode human variant ACE2 extracellular domains. The ACE2 nucleotides can be joined together with nucleotides encoding a polypeptide that can inhibit the complement pathway and/or a polypeptide that prolongs half-life of the fusion protein in the blood and/or serum. These joined polynucleotides will encode fusion proteins having an ACE2 portion and a portion encoding the complement inhibiting protein and/or the polypeptide portion that prolongs blood and/or serum half-life. Complement inhibitors that can be used in the fusions with the ACE2 portion include, for example, CR1 SCR1-3 (SEQ ID NO:9-10), CR1 SCR1-3 N29K/S37Y/G79N/D109N (SEQ ID NO:11-12), DAF SCR2-4 (SEQ ID NO:13-14), MCP SCR2-4 (SEQ ID NO:15-16), Factor H SCR1-4 (SEQ ID NO:17-18), and C4BPA SCR1-3 (SEQ ID NO: 19-20). Polypeptides that can enhance serum and/or blood half-life include, for example, albumin, IgG4 Fc, or COL18A1 trimeric domain.

The polynucleotides may be operatively linked to one or more control sequences that control gene expression to create a recombinant polynucleotide capable of expressing the polypeptide. Expression constructs containing a heterologous polynucleotide encoding the engineered ACE2 molecules can be introduced into appropriate host cells to express the corresponding ACE2 molecule.

The polynucleotide can encode an engineered ACE2 molecule and can have at least 60%, 65%, 70%, 75%, 80%, 85%, 90%, 91%, 92%, 93%, 94%, 95%, 96%, 97%, 98%, 99% or more sequence identity to a reference sequence selected from: SEQ ID NO: 1, 3, 5, and 7. The polynucleotide can encode an engineered ACE2 molecule and can hybridize under stringent hybridization conditions to a nucleic acid having the sequence of one of SEQ ID NO: 1, 3, 5, and 7, or a complement thereof. The polynucleotide encoding the fusion partner of the ACE2 molecule and can have at least 60%, 65%, 70%, 75%, 80%, 85%, 90%, 91%, 92%, 93%, 94%, 95%, 96%, 97%, 98%, 99% or more sequence identity to a reference sequence selected from: SEQ ID NO: 9, 11, 13, 15, 17, 19, 21, 23, or 25. The polynucleotide can encode an engineered ACE2 molecule and can hybridize under stringent hybridization conditions to a nucleic acid having the sequence of one of SEQ ID NO: 9, 11, 13, 15, 17, 19, 21, 23, or 25, or a complement thereof.

The polynucleotides can be codon optimized to fit the host cell in which the protein is being produced. For example, preferred codons used in bacteria are used to express the gene in bacteria; preferred codons used in yeast are used for expression in yeast; and preferred codons used in mammals are used for expression in mammalian cells.

The control sequences can include among others, promoters, enhancers, leader sequences, polyadenylation sequences, propeptide sequences, signal peptide sequences, and transcription terminators. Other control sequences will be apparent to the person of skill in the art.

Suitable promoters can be selected based on the host cells used. For bacterial host cells, suitable promoters for directing transcription of the nucleic acid constructs of the present disclosure, include the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene, the tac promoter, or the T7 promoter.

Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase.

Exemplary promoters for insect cells include, among others, those based on polyhedron, PCNA, OplE2, OplE1, Drosophila metallothionein, and Drosophila actin 5C. In some embodiments, insect cell promoters can be used with Baculoviral vectors.

Exemplary promoters for plant cells include, among others, those based on cauliflower mosaic virus (CaMV) 35S, polyubiquitin gene (PvUbi1 and PvUbi2), rice (Oryza sativa) actin 1 (OsAct1) and actin 2 (OsAct2) promoters, the maize ubiquitin 1 (ZmUbi1) promoter, and multiple rice ubiquitin (RUBQ1, RUBQ2, rubi3) promoters.

Exemplary promoters for mammalian cells include, among others, CMV IE promoter, elongation factor 1a-subunit promoter, ubiquitin C promoter, Simian Virus 40 promoter, and phosphoglycerate Kinase-1 promoter.

The control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used.

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used herein.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region which directs the expressed polypeptide into the secretory pathway of a host cell of choice may be used in the present disclosure.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide can be converted to a mature active polypeptide by catalytic or autocatalytic cleavage of the propeptide from the propolypeptide. Where both signal peptide and propeptide regions are present at the amino terminus of a polypeptide, the propeptide region is positioned next to the amino terminus of a polypeptide and the signal peptide region is positioned next to the amino terminus of the propeptide region.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter.

In another aspect, the present disclosure is also directed to a recombinant expression vector comprising a polynucleotide encoding a polypeptide described herein, and one or more expression regulating regions such as a promoter and a terminator, a replication origin, etc., depending on the type of hosts into which they are to be introduced.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The expression vector can exist as a single copy in the host cell, or maintained at higher copy numbers, e.g., up to 4 for low copy number and 50 or more for high copy number.

In some embodiments, the expression vector contains one or more selectable markers, which permit selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol (Example 1) or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

### Host Cells

The host cell may be, for example, a bacterium, a yeast or other fungal cell, insect cell, a plant cell, or a mammalian cell. Exemplary prokaryote host cells include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P), Pseudomonas such as P. aeruginosa, and Streptomyces. These examples are illustrative rather than limiting. Strain W3110 is one particularly preferred host or parent host because it is a common host strain for recombinant polynucleotide product fermentations. Preferably, the host cell secretes minimal amounts of proteolytic enzymes. For example, strain W3110 may be modified to effect a genetic mutation in the genes encoding polypeptides endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan′; E. coli W3110 strain 37D6, which has the complete genotype tonA ptr3 phoA E15 (argF-lac)169 degP ompT rbs7 ilvG kan′; E. coli W3110 strain 40B4, which is strain 37D6 with a non-kanamycin resistant degP deletion mutation; and an E. coli strain having mutant periplasmic protease. Alternatively, in vitro methods of cloning, e.g., PCR or other nucleic acid polymerase reactions, are suitable.

Eukaryotic microbes may be used for expression. Eukaryotic microbes such as filamentous fungi or yeast are suitable cloning or expression hosts for polypeptide-encoding vectors. Saccharomyces cerevisiae is a commonly used lower eukaryotic host microorganism. Others include Schizosaccharomyces pombe; Kluyveromyces hosts such as, e.g., K. lactis (MW98-8C, CBS683, CBS4574), K. fragilis (ATCC 12,424), K bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans, and A. niger. Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. Saccharomyces is a preferred yeast host, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian tissue cell culture may also be used to express and produce the polypeptides as described herein and in some instances are preferred (See Winnacker, From Genes to Clones VCH Publishers, N.Y., N.Y. (1987). For some embodiments, eukaryotic cells may be preferred, because a number of suitable host cell lines capable of secreting heterologous polypeptides (e.g., intact immunoglobulins) have been developed in the art, and include CHO cell lines, various Cos cell lines, HeLa cells, preferably, myeloma cell lines, or transformed B-cells or hybridomas. The mammalian host cell can be a CHO cell.

Examples of other useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/− DHFR(CHO or CHO-DP-12 line); mouse sertoli cells; monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TM cells; MRC 5 cells; FS4 cells; and a human hepatoma line (Hep G2).

### Pharmaceutical Compositions

Pharmaceutical compositions of the present invention may comprise a ACE2 molecule, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions may be administered in a manner appropriate to the disease to be treated (or prevented). The quantity and frequency of administration will be determined by such factors as the condition of the patient, and the type and severity of the patient's disease, although appropriate dosages may be determined by clinical trials.

Suitable pharmaceutically acceptable excipients are well known to a person skilled in the art. Examples of the pharmaceutically acceptable excipients include phosphate buffered saline (e.g. 0.01 M phosphate, 0.138 M NaCl, 0.0027 M KCl, pH 7.4), an aqueous solution containing a mineral acid salt such as a hydrochloride, a hydrobromide, a phosphate, or a sulfate, saline, a solution of glycol or ethanol, and a salt of an organic acid such as an acetate, a propionate, a malonate or a benzoate. An adjuvant such as a wetting agent or an emulsifier, and a pH buffering agent can also be used. The pharmaceutically acceptable excipients described in Remington's Pharmaceutical Sciences (Mack Pub. Co., N.J. 1991) (which is incorporated herein by reference in its entirety for all purposes) can be appropriately used. The composition can be formulated into a known form suitable for parenteral administration, for example, injection or infusion. The composition may comprise formulation additives such as a suspending agent, a preservative, a stabilizer and/or a dispersant, and a preservation agent for extending a validity term during storage.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, intranasally, intraarterially, intratumorally, into an afferent lymph vessel, by intravenous (i.v.) injection, or intraperitoneally. In one aspect, the ACE2 molecule compositions of are administered to a patient by intradermal or subcutaneous injection. In one aspect, the ACE2 molecule compositions of are administered by i.v. injection.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1 Expression of ACE2 Fusion (AF) Proteins

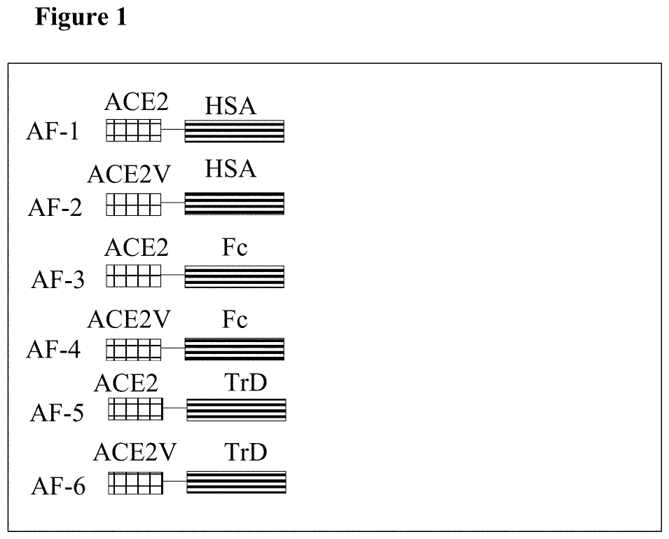

cDNA of the human ACE2 extracellular domain is synthesized and fused with the HSA, the human immunoglobulin Fc domain, or the human collagen trimeric domain (AF-1, AF-3, or AF-5, FIG. 1). The ACE2 could be the enzymatically inactive variant ACE2V (AF-2, AF-4, or AF-6, FIG. 1). The fusion proteins are cloned into a mammalian expression vector under the control of a hEF1α promoter. A linker maybe inserted between the domains. The vector contains a Puromycin resistant gene for mammalian cell selection and an Ampicillin resistant gene for E. coli propagation. All fusion proteins contained a signal peptide at the N-terminal for secretion out of the cells. The expression vector plasmids are used to transfect 100 ml of 293 cells transiently. The culture media is harvested after 72 hours and the fusion protein is purified.

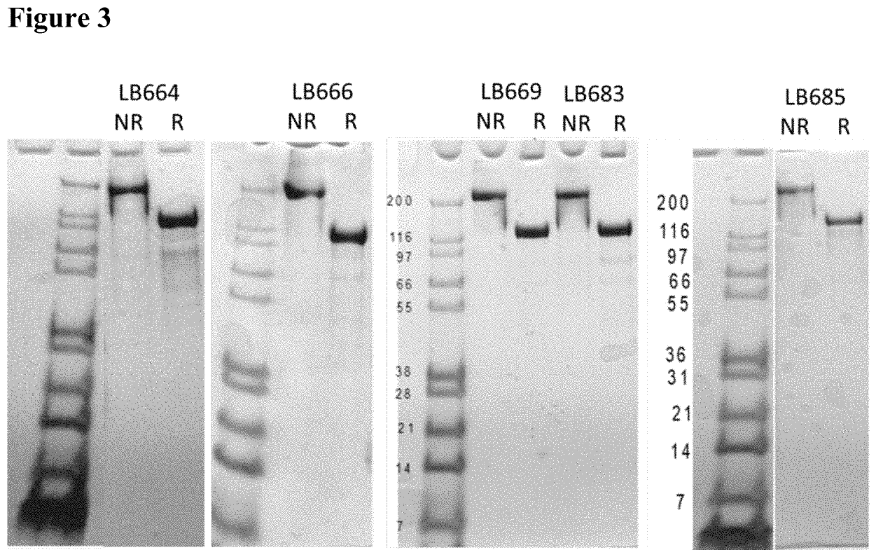

Various human ACE2 extracellular domains (ECD) have been produced as Fc fusion proteins. LB701 contains the full length ACE2 ECD (SEQ ID NO 2); LB664 contains the ACE2 ECD with a deletion (SEQ ID NO 4); LB666 contains the enzymatically inactive ACE2(H374N/H378N) (SEQ ID NO 8). They were purified by Protein A chromatography after transient transfection in 293 cells or stable transfection in CHO cells. Purified LB664 and LB664 are shown in FIG. 3.

Amino acid residues important for binding of ACE2 and SARS-CoV-2 spike proteins were identified from the published crystal structures (Lan 2020, Shang 2020, Yan 2020). Point mutations predicted to enhance binding of the spike proteins were introduced in LB664 to make LB697 vectors which express the ACE2 variants as Fc fusion proteins. After charactering the binding properties of the single mutations, multiple mutations were generated in the ACE2-Fc (Table 2). LB801, LB802, LB803, LB804, LB805, and LB806 contain the full length ACE2 ECD (SEQ ID NO 2) and the D30E/Q42K/L79W/N330Y, T27Y/D30E/Q42K/L79W/N330Y, T27R/D30E/Q42K/L79W/N330Y, K31F N33D H34 S E35Q, D30E/K31F/H34I/E35Q/Q42K/L79W/N330Y, and D30E/H34V/Q42K/L79W/N330Y mutations, respectively.

TABLE 2
ACE2-Fc variants produced and characterized

| | |
|---|---|
| Single | K26E, K26R, T27R, F28W, D30E, K31E, Y41N, Q42E, |
| mutation | L45E, L79W, Y83F, G326E, N330K, N330Q, N330Y, |
| | G352Y, K353H |
| 2 | K26R/L79W, T27R/L79W, F28W/L79W, D30E/L79W, |
| mutations | Q42E/L79W |
| 3 | K26R/D30E/L79W, D30E/L79W/N330Q, D30E/L79W/ |
| mutations | N330Y, D30E/Q42K/L79W |
| 4 | K26R/F28W/D3 0E/L79W, D30E/Q42K/L79W/N33OQ, |
| mutations | D30E/Q42K/L79W/N330Y, K31F_N33D_H34S_E35Q |
| 5 | K26R/T27R/F28W/D30E/L79W, T27R/D30E/Q42K/L79W/ |
| mutations | N330Y, T27Y/D30E/Q42K/L79W/N330Y, D30E/H34V/ |
| | Q42K/L79W/N330Y |
| 7 | D30E/K31F/H34I/E35Q/Q42K/L79W/N330Y |
| mutations | |

### Example 2 Expression of ACE2 and Complement Inhibitor Fusion (ACF) Proteins

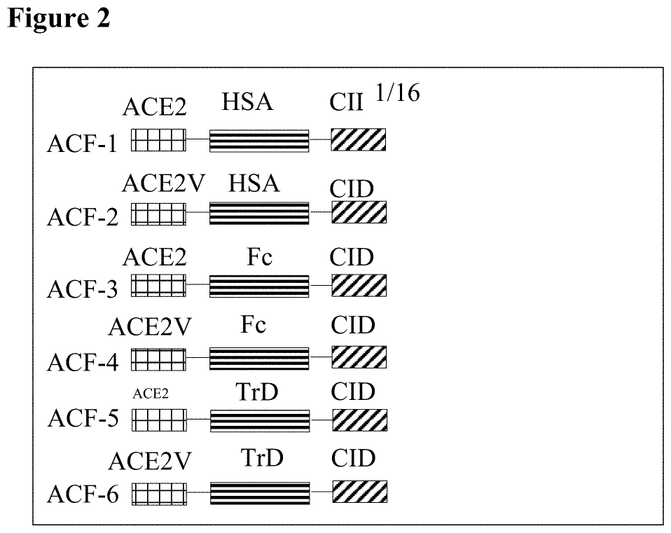

cDNA of the human CR1 SCR1-3 N29K/S37Y/G79D/D109N is synthesized and fused at the C-terminus of AF-1-6 to make ACF-1-6 (FIG. 2). A linker maybe be inserted before the CR1 domain. The vector contains a Puromycin resistant gene for mammalian cell selection and an Ampicillin resistant gene for E. coli propagation. All fusion proteins contained a signal peptide at the N-terminal for secretion out of the cells. The expression vector plasmids are used to transfect 100 ml of 293 cells transiently. The culture media is harvested after 72 hours and the fusion protein is purified.

Various ACF fusion proteins have been produced. LB669 contains the functional ACE2 ECD with a deletion (SEQ ID NO 4); LB683 contains the enzymatically inactive ACE2(H374N/H378N) (SEQ ID NO 8); LB684 contains the enzymatically inactive ACE2 (R514Q) (SEQ ID NO 6). The CIDv9 domain was also fused at the N terminus of ACE2-Fc in LB664 to make LB685. LB771 and LB772 contain the functional ACE2 ECD with a deletion (SEQ ID NO 4) and the affinity enhanced D30E/L79W/N330Y and D30E/Q42K/L79W/N330Y mutations, respectively; LB833, LB834, and LB835 contain the full length ACE2 ECD (SEQ ID NO 2) and the T27Y/D30E/Q42K/L79W/N330Y, T27R/D30E/Q42K/L79W/N330Y, and D30E/Q42K/L79W/N330Y mutations, respectively. They were purified by Protein A chromatography after transient transfection in 293 cells or stable transfection in CHO cells. Purified LB669, LB683 and LB685 are shown in FIG. 3.

### Example 3 In Vitro Binding of the ACE2 Fusion Protein to SARS-COV-2 Spike Protein

The ability of the soluble ACE2 fusion proteins AFs and ACFs to bind to the S1 domain of the SARS-COV-2 spike protein is determined in a functional ELISA. Briefly Spike protein S1 domain (Sino Biological) is coated on standard ELISA plates at 2.5 μg/mL, then incubated overnight at 4° C. The wells are blocked for an hour at room temperature. Dilutions of the ACE2 fusion proteins are added to the plates and incubated for an hour at 37° C. The wells are washed, and bound ACE2 fusion protein is detected using polyclonal goat anti-ACE2 IgG (R&D Systems) and reported with donkey anti-goat IgG labeled with HRP. After washing, TMB reagent (Sigma) is added and OD absorption at 450 nm is measured in a plate reader.

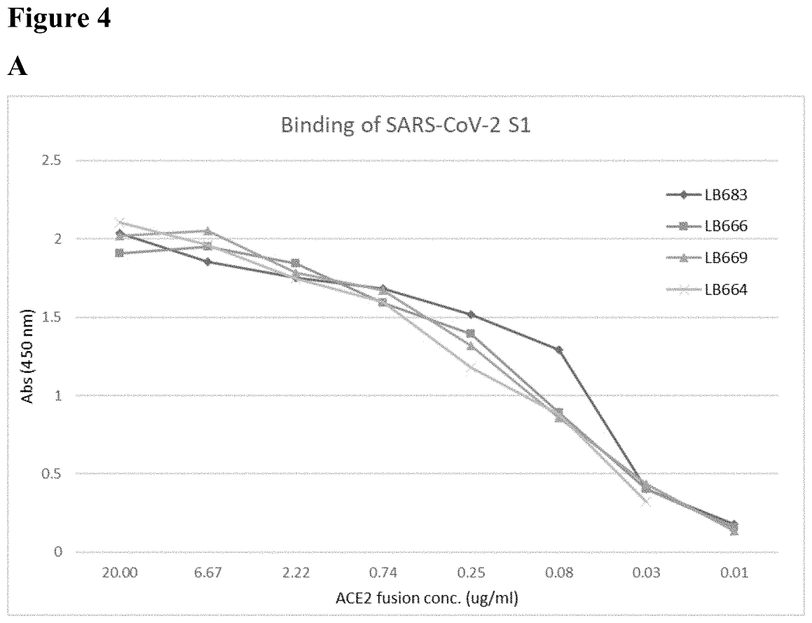

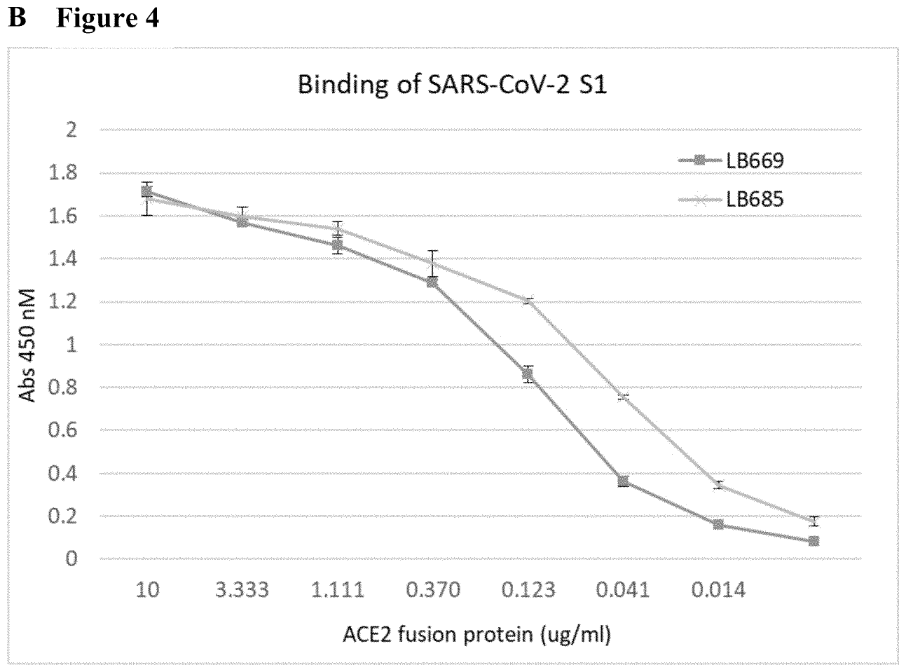

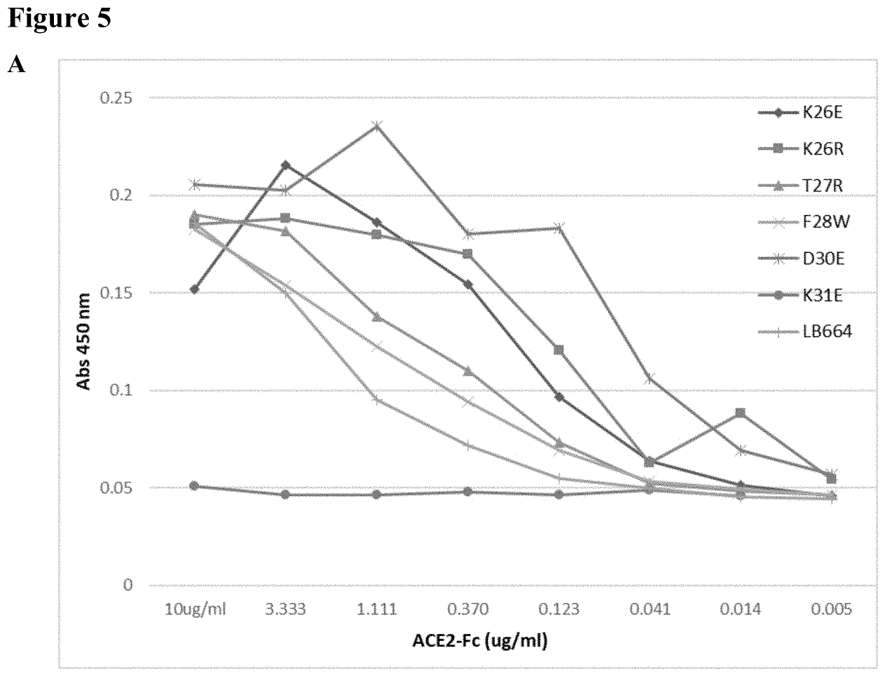

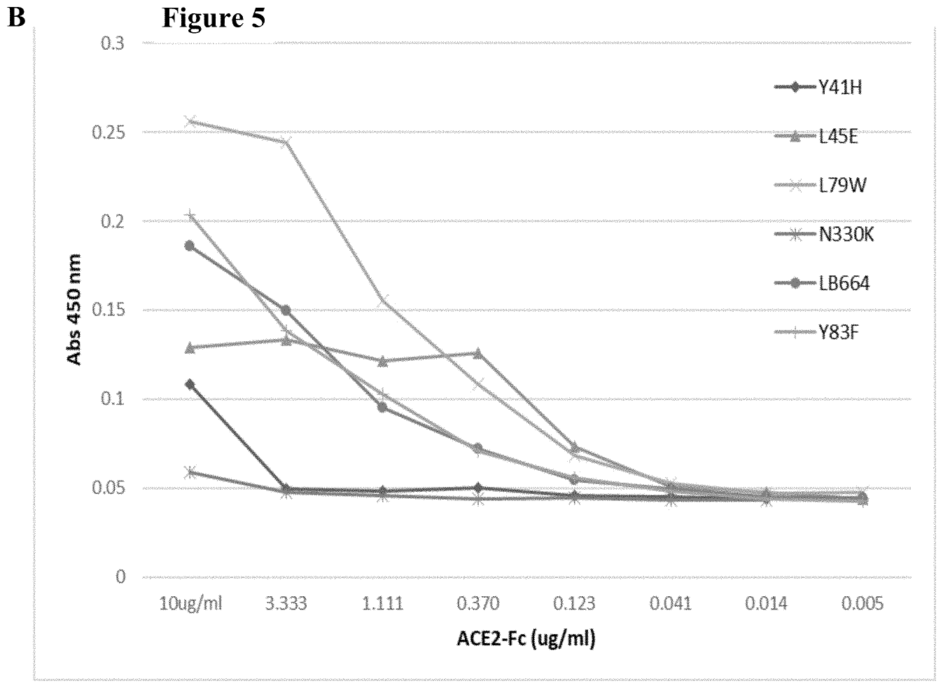

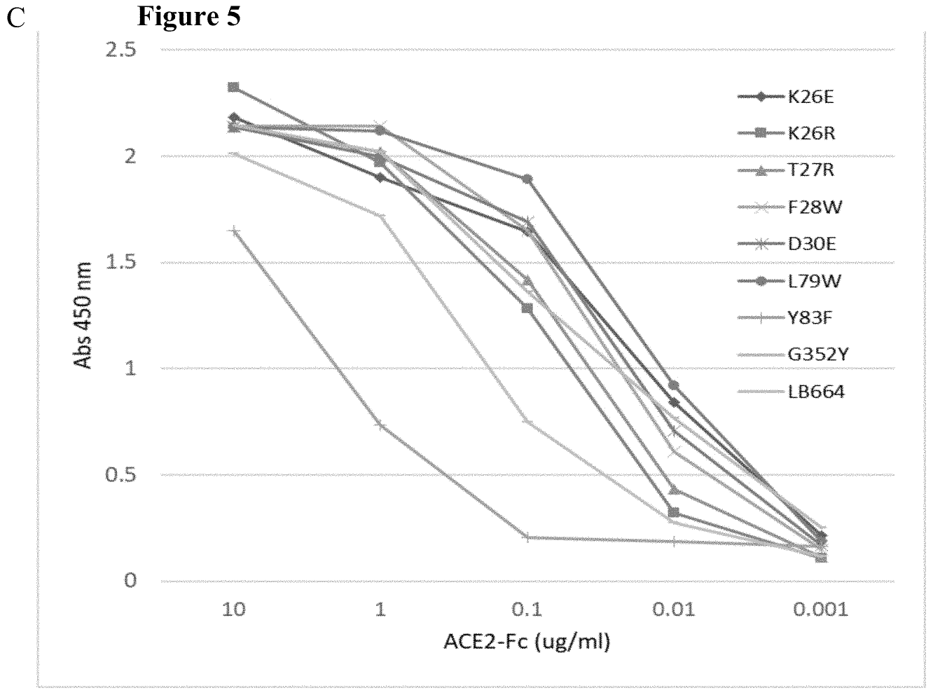

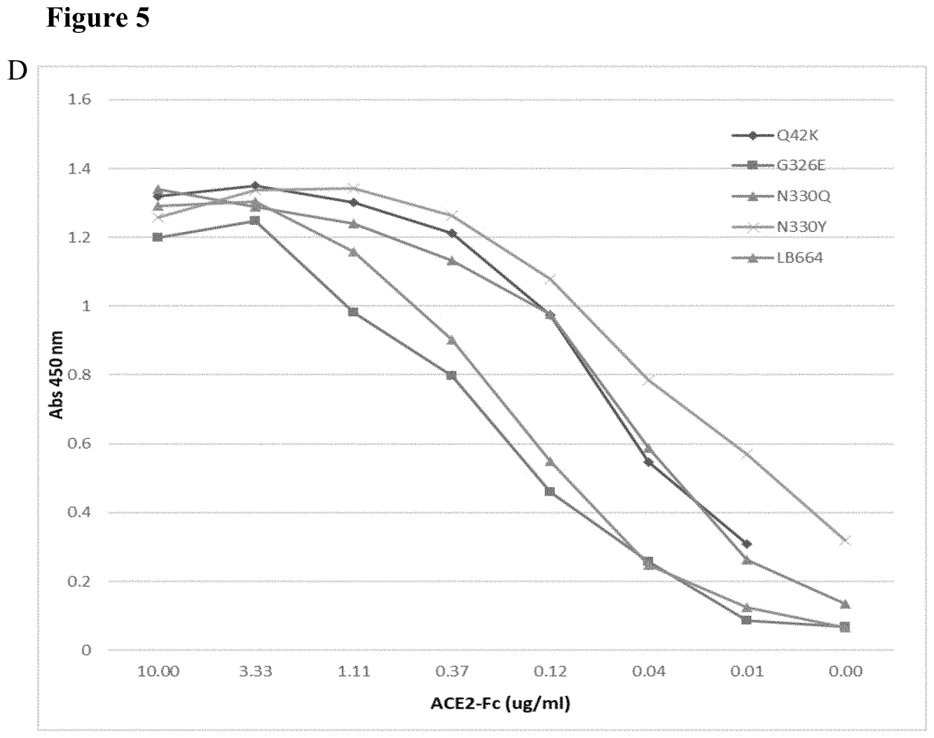

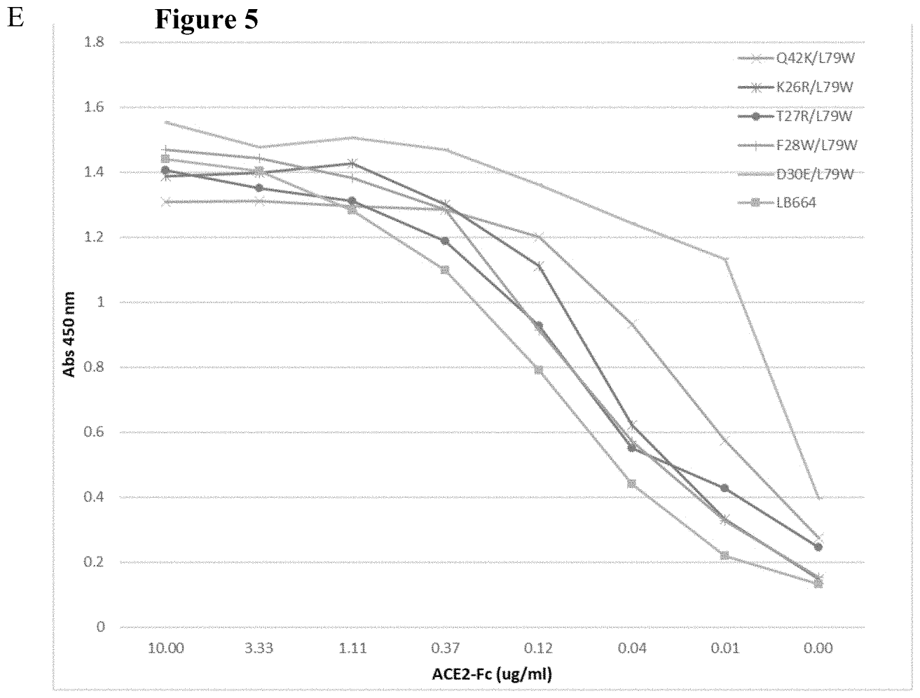

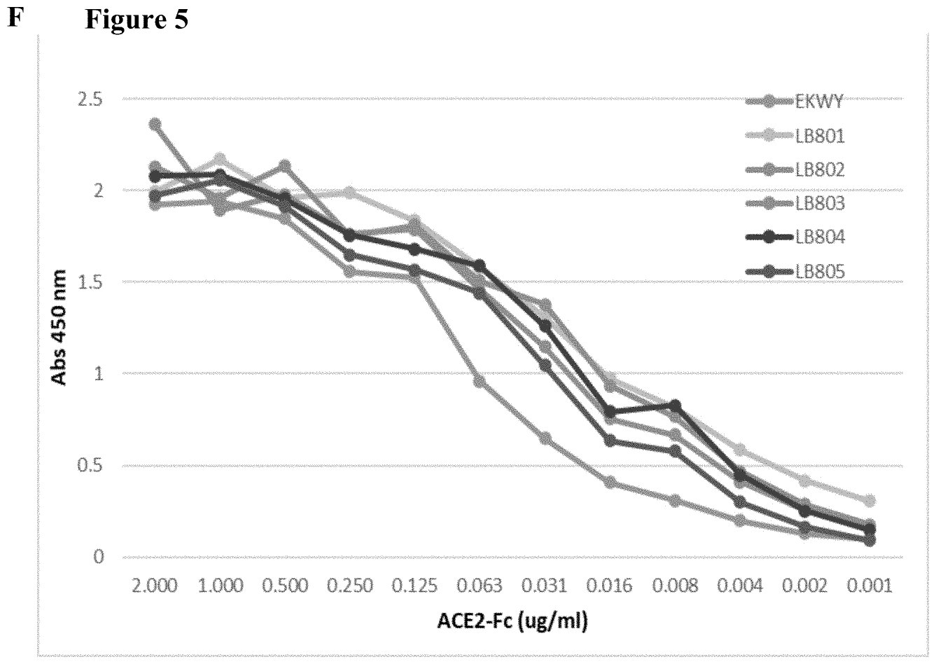

The SARS-CoV2 spike protein (2 μg/ml) was coated in an ELISA plate. After blocking with 1% BSA, 3-fold dilution series of LB664, LB666, LB669, and LB683 starting from 20 μg/ml were added to the ELISA plate. After washing, the bound ACE2 fusion protein was detected with anti-human Fc HRP. All 4 ACE2 fusion proteins exhibited similar binding affinities to the spike protein (FIG. 4A). LB669 and LB685 exhibited similar binding activities in a separate ELISA assay (FIG. 4B). Binding activities of the ACE2 variants (Table 2) were characterized similarly and compared with LB664. Single mutations of K26E, K26R, T27R, F28W, D30E, Q42K, L79W, G352Y, N330Q, and N330Y enhanced binding of the SARS-CoV2 spike protein (FIG. 5A-D). Some of the double mutations including K26R/L79W, T27R/L79W, F28W/L79W, D30E/L79W, and Q42K/L79W enhanced the binding activities as well (FIG. 5E). The binding kinetics of multiple ACE2 variants with the RBD domain of SARS-CoV2 spike protein were characterized on Octet (Table 3). The ACE2-Fc with quadruple mutations of D30E/Q42K/L79W/N330Y exhibited the highest affinity of 0.91 nM. LB801 containing the D30E/Q42K/L79W/N330Y mutations in the full length ACE2 ECD (SEQ ID NO 2) exhibited a higher binding affinity to the SARS-CoV2 spike protein than LB697-D30E/Q42K/L79W/N330Y which contains the C-terminal deletion of ACE2 ECD (SEQ ID NO 4) (FIG. 5F). LB802-805 also exhibited potent binding affinities (FIG. 5F).

TABLE 3
Binding kinetics of engineered ACE2-Fc variants

| | KD | Kon | Koff |
|---|---|---|---|
| | (nM) | (1/Ms) | (1/s) |
| LB664 | 31.8 | 3.85E+05 | 1.22E−02 |
| LB697-L79W | 8.52 | 7.21E+05 | 6.14E−03 |
| LB697-D30E/L79W | 4.91 | 7.27E+05 | 3.57E−03 |
| LB697-D30E/L79W/N330Y | 1.49 | 8.08E+05 | 1.20E−03 |
| LB697-D30E/Q42K/L79W/N330Y | 0.91 | 8.10E+05 | 7.40E−04 |

### Example 4 Inhibition of the Classical Complement Pathway by ACFs

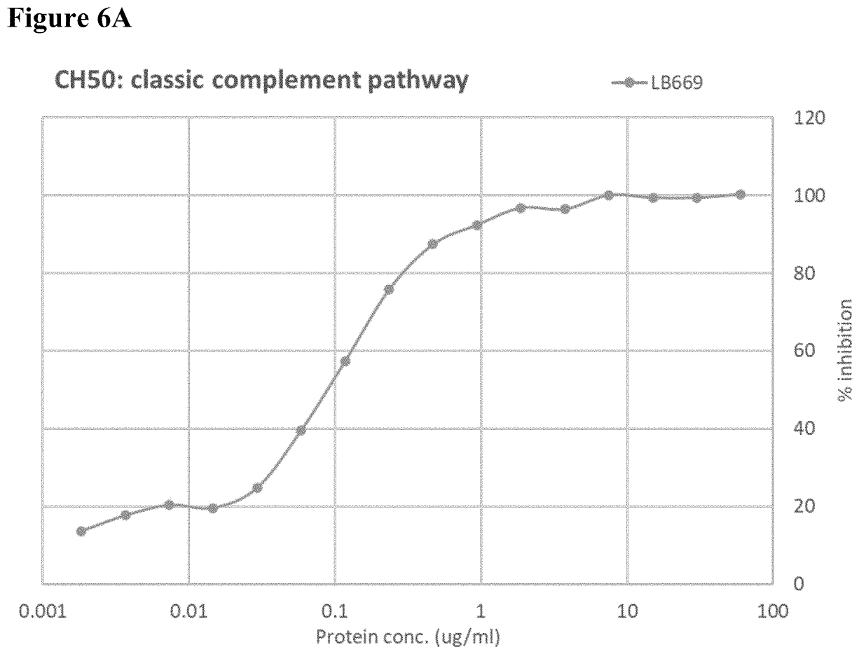

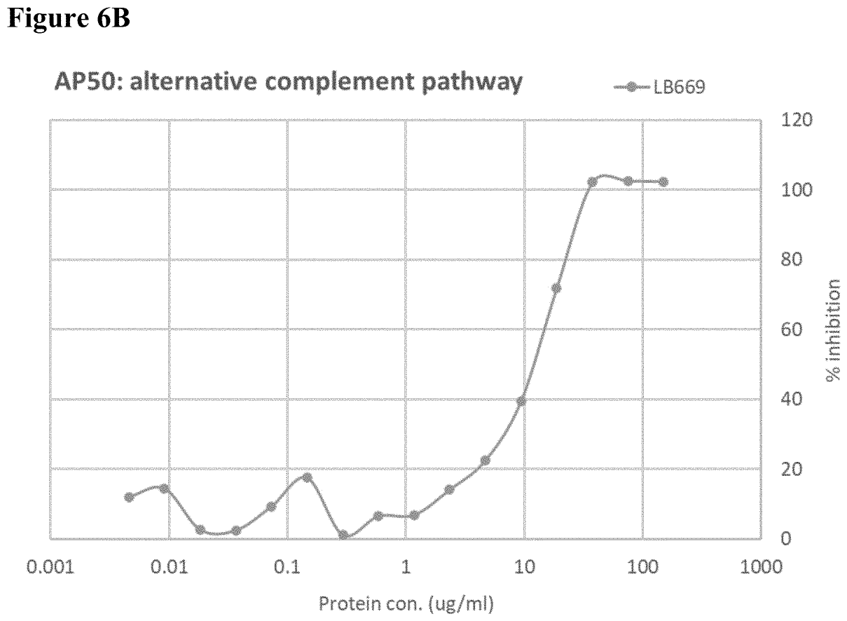

The classical pathway activity CH50 assay (Kabat, 1961) measures the ability of a sample to lyse 50% of a standardized suspension of sheep erythrocytes coated with anti-erythrocyte antibody (EA, antibody sensitized sheep erythrocytes, Complement Technology). The dilution of the normal human serum (Complement Technology) that lyses 90% of 1×10E7 EA/mL after 1 h incubation at 37° C. is first determined. The assay is carried out in GVB⁺⁺ buffer (0.1% gelatin, 5 mM Veronal, 145 mM NaCl, 0.025% NaN₃, pH 7.3) containing 0.15 mM CaCl₂) and 0.5 mM MgCl₂. Inhibition of the classical complement pathway is activated by mixing the dilution of normal human serum that should lyse 90% of EA with 0-500 nM of the testing proteins for 1 hour at 37° C. Hemolysis of EA is then assayed after 1 hour incubation of the serum and EA by measuring OD at 541 nm. LB669 exhibited a potent inhibition of the classical complement pathway with EC50 of ˜0.1 μg/ml (FIG. 6A).

### Example 5 Inhibition of the Alternative Complement Pathway by ACFs

In contrast to the classical complement pathway, activation of the alternative complement pathway requires only magnesium ions but not calcium ions. Thus, the assay described above is modified to contain 5 mM Mg⁺ and 5 mM EGTA, which preferentially chelates calcium ions. For this assay, the dilution of normal human serum (Complement Technology) that lyses 90% of 1.25×10E7 rabbit erythrocytes/mL (Er, Complement Technology) is first determined after 30 minutes incubation at 37° C. The assay is performed in GVB⁰ buffer (0.1% gelatin, 5 mM Veronal, 145 mM NaCl, 0.025% NaN₃, pH 7.3) containing 5 mM MgCl₂ and 5 mM EGTA. Inhibition of the alternative complement pathway is initiated by mixing the dilution of normal human serum that should lyse 90% of Er with 0-500 nM of the testing proteins for 1 hour at 37° C. Hemolysis of Er is then assayed after 30 minutes incubation of the serum and Er by measuring OD at 412 nm. LB669 exhibited a robust inhibition of the alternative complement pathway with EC50 of ˜13.1 μg/ml (FIG. 6B).

### Example 6 Characterization of the Peptidase Activity of the ACE2 Fusion Proteins

The peptidase activity of the ACE2 fusion proteins is assessed with a fluorogenic assays using the synthetic ACE2 substrate, Mca-APK(Dnp) (Enzo Life Sciences). The assay is carried out at room temperature and monitored continuously by measuring the increase in fluorescence (excitation ¼ 340 nm, emission ¼ 430 nm) upon substrate hydrolysis in a plate reader. Initial velocities are determined from the linear rate of fluorescence increase over the 0-60 min time course. The reaction product is quantified by using standard solutions of Mca.

The peptidase activities of LB664, LB666, LB669, and LB683 were assessed with the recombinant ACE2 (R&D system) as the positive control. LB664 and LB669 exhibited similar activities as the positive control, whereas LB666 and LB683 were enzymatically inactive as expected (Table 4). The enzymatical activities of all the affinity-enhanced ACE2 variants to the SARS-CoV2 spike protein have also been confirmed.

TABLE 4
Peptidase activities of the ACE2 fusion proteins

| | | Peptidase Activity |
|---|---|---|
| Protein | Structure | (pmol/min/μg ACE2) |
| ACE2 | Recombinant (R&D) | 800 |
| LB664 | ACE2-Fc | 732 |
| LB666 | ACE2(H374N/H378N)-Fc | 22 |
| LB683 | ACE2(H374N/H378N)-Fc-CIDv9 | 0 |
| LB669 | ACE2-Fc-CIDv9 | 745 |
| LB685 | CIDv9-ACE2-Fc | 733 |

### Example 7 Inhibition of Viral Infection in a Pseudotyped Viral Entry Model

The ability of ACFs to block viral entry is assessed using pseudotyped SARS-CoV-2 S virions and VeroE6 cells, which are known to express ACE2 and to support SARS-CoV and SARS-CoV-2 replication.

MLV-based SARS-CoV-2 S pseudotype is prepared as described by Millet and Whittaker, 2016. HEK293T cells are co-transfected using Lipofectamine 2000 (Life Technologies) with an S encoding-plasmid, an MLV Gag-Pol packaging construct, and the MLV transfer vector encoding a luciferase reporter, according to the manufacturer's instructions. Cells are incubated for 5 h at 37° C. with transfection medium. Cells are then washed with DMEM two times and then DMEM containing 10% FBS is added and incubated for 60 h. The supernatants are then harvested and filtered through 0.45-um membranes, concentrated by centrifugation using a 30-kDa cut-off membrane for 10 min. at 3,000 rpm and then frozen at −80° C.

VeroE6 cells are cultured in Dulbecco's Modified Eagle Medium (DMEM) containing 10% fetal bovine serum (FBS) and 1% PenStrep. VeroE6 cells are plated into 12-well plates at a density of 0.3×10E6 and incubated at 37 C for 16 h. 20 uL of concentrated pseudovirus is added to the wells after washing three times with DMEM. In some experiments, ACF is added before or during incubation with pseudovirus. After 2-3 h, DMEM with 20% FBS and 2% PenStrep is added to the cells, which are then cultured for 48 h. Following the 48-h infection, One-Glo-EX (Promega) was added to the cells and incubated in the dark for 10 min. prior to reading luminescence on an EnVision MultiLabel Reader (Perkin Elmer). Results demonstrate that viral entry is inhibited by various ACFs.

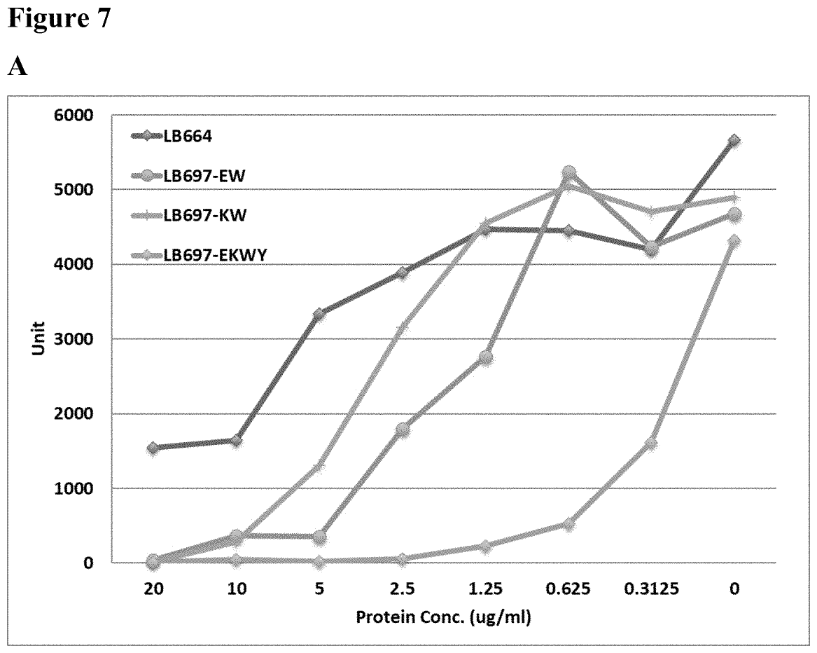

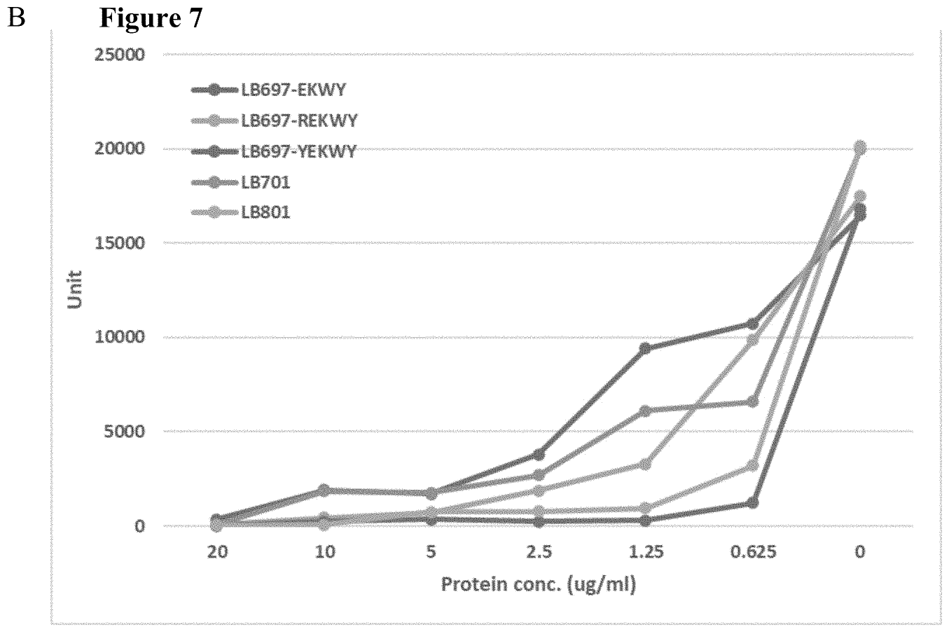

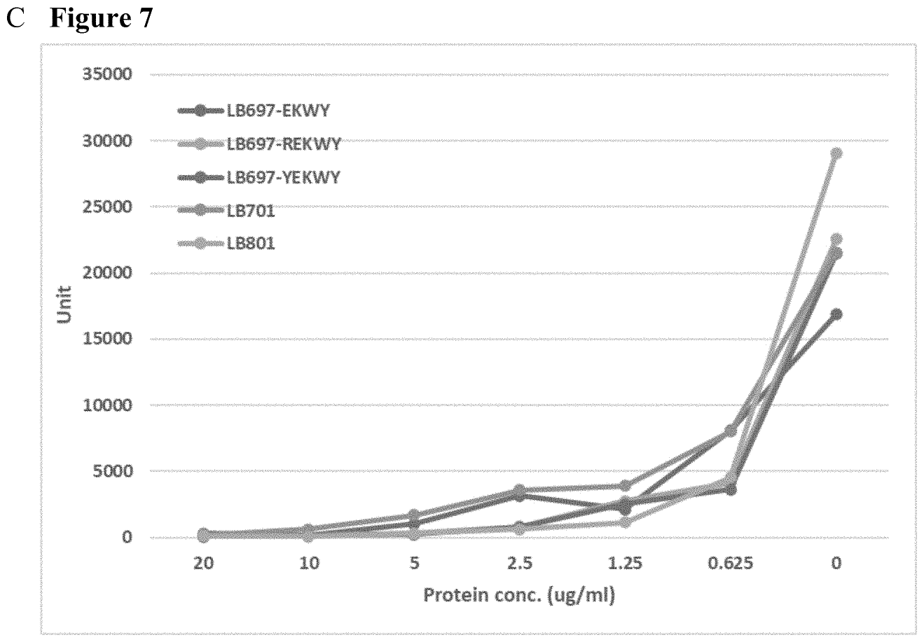

Pseudotyped SARS-CoV2 was constructed with a lentiviral expression vector LB686 expressing a luciferase reporter gene and LB733 expressing the SARS-CoV2 spike protein. LB686, LB733, and the lentiviral packaging vector were co-transfected in HEK293 cells. The lentivirus was harvested after 4 days, and used to infect HEK293 cells expressing human ACE2. The infection activity was characterized by assaying the luciferase activity in the cell lysate after 2 days. To test the blocking activities of the ACE2 fusion proteins to the viral infection, they were premixed with the virus prior to the infection. LB664 inhibited the pseudotyped SARS-CoV2 infection with a modest activity with EC50 of 5-10 μg/ml. The high affinity variants of ACE2 fusions LB697-EW (D30E/L79W), LB697-KW (Q42K/L79W), LB697-EKWY (D30E/Q42K/L79W/N330Y) exhibited higher blocking activities, with EC50s of ˜1, 3, 0.3 μg/ml respectively (FIG. 7A). The ACE2 variants with the full length ECD exhibited better blocking activities of the viral infection (FIG. 7B).

Pseudotyped SARS-CoV2 with the spike protein of the South Africa variant 501Y.v2 was produced similarly with the lentiviral expression system. The spike protein was cloned into an expression vector LB798. LB686, LB798, and the lentiviral packaging vector were co-transfected in HEK293 cells. The lentivirus was harvested after 4 days, and used to infect HEK293 cells expressing human ACE2. The ACE2 fusion proteins exhibited potent blocking activities of the viral infection (FIG. 7C).

### Example 8 Pharmacokinetic Assessment of ACFs in Mice

10 mg/kg of ACFs is administered into mice intravenous injection. Serum samples are taken at different time points after the injection up to 15 days. Concentrations of the fusion protein in the serum samples are determined using a sandwiched ELISA assay.

### Example 9 Characterization of the Protective Activity of ACFs in Acid-Induced ALI Models

Acid aspiration in mice, which mimics human acute lung injury/ARDS, results in rapid impairment of lung functions assessed by increased lung elastance (a measure of the change in pressure achieved per unit change in volume, representing the stiffness of the lungs), decreased blood oxygenation and the development of pulmonary oedema. The protective activities of ACFs are evaluated in the acid-induced ALI model.

3 month old mice are anaesthetized with ketamin (75 mg/kg) and xylazine (20 mg/kg) i.p., tracheostomized and ventilated with a volume control constant flow ventilator (Voltek Enterprises). Volume recruitment manoeuvre (VRM) (25 cmH₂O, 3 sec) is performed to standardize volume history and measurements are made as baseline. 30 min before the acid instillation, the mice are administered various ACFs. After intratracheal instillation of HCl (pH=1.5; 2 ml/kg), followed by a VRM (35 cmH₂O, 3 seconds), animals are ventilated for 3 hrs (F₁O₂ 1.0). Saline-treated groups served as controls.

Total PEEP (PEEPt) and plateau pressure (Pplat) are measured at the end of expiratory and inspiratory occlusion, respectively, and elastance is calculated as Pplat minus PEEPt)/VT every 30 minutes during the ventilation periods. At the end of the ventilation, left lungs are sampled for the measurement of lung wet/dry mass ratios or snap frozen in liquid nitrogen for subsequent biochemical analysis, and right lungs are fixed in 10% buffered formalin for histological examination.

At the end of the experiments, blood samples are obtained from the left heart ventricle and PaO₂ is measured (Ciba-Corning Model 248) to assess arterial blood oxygenation as an indicator for respiratory failure. To assess pulmonary oedemas, the lung wet/dry weight ratios are calculated. In brief, after the blood is drained from the excised lungs, measurements of the lung wet weight are made. Lungs are then heated to 65° C. in a gravity convection oven for 24 hrs and weighed to determine baseline lung dry mass levels. Pulmonary vascular permeability is assessed by measuring the pulmonary extravasation of Evans Blue. Evans Blue (20 mg/g) is injected into the jugular vein at the end of the 3 hrs ventilation period. Ten minutes after the injection of Evans Blue, the animals are sacrificed. Lungs are then perfused with ice-cold PBS before the lung tissue is used to determine the content of Evans Blue.

### Example 10 Characterization of the Protective Activity of ACFs in a SARS-CoV Infection Model

C57BL/6J (Jackson Laboratories) mice (10- to 11-week-old) are anesthetized with a mixture of ketamine-xylazine and intranasally inoculated with 50 μl of phosphate-buffered saline (PBS) or 10E5 PFU of SARS-CoV diluted in PBS. Mice are monitored for disease signs and weighed at 24-h intervals. Mice are sacrificed at 3, 12, 24, 48, and 72 h post-infection for assessment of viral replication, lung pathology, and complement activation. ACFs (e.g. range of 0.5-50 mg/kg) are given prior to, with and/or at various times after viral inoculation.

For each mouse, lung samples are weighed and homogenized in five volumes of PBS to give a 20% solution. The solution is centrifuged at 13,000 rpm under aerosol containment in a tabletop centrifuge for 5 min. The clarified supernatant is serially diluted in PBS, and 200 uL of each dilution is applied to monolayers of Vero E6 cells in six-well plates. Following 1 hour of incubation at 37° C., the cells are overlaid with 0.8% agarose-containing medium. Two days later, the plates are stained with neutral red, and the plaques are counted.

Multiple tissues obtained from necropsy are fixed in 10% buffered formalin for 72 h, transferred to 70% ethanol, and later paraffin embedded. Histopathologic evaluation is performed on deparaffinized sections stained by routine hematoxylin-and-eosin staining. Histology shows reduced damage to lungs and other tissues following viral infection when ACFs are given to mice.

Immunohistochemical (IHC) testing for SARS-CoV is applied using a colorimetric indirect immunoalkaline phosphatase method with a rabbit anti-SARS-CoV nucleocapsid protein antibody (Imgenex). Results indicate lower levels of SARS-CoV nucleocapsid protein, indicating lower viral replication in mice treated with ACF prior to or after infection.

Lung sections from SARS-CoV or mock-infected mice are stained for the presence of C3. Lung samples are tested from 20-week-old mice at 1, 2, 4, and 7 days after infection with 10E5 PFU of virus. Staining is performed using a goat anti-mouse C3 primary antibody (MP Biomedicals). Complement activation is attenuated in mice treated with an ACF.

### Example 11 Characterization of the Protective Activity of ACFs in a Murine Coronaviral Infection Model

Transgenic mice expressing human ACE2 (huACE2) under the control of the CAG promotor are generated by standard methods. Anesthetized huACE2 transgenic mice and their nontransgenic littermates at the ages of 8 to 20 weeks are inoculated via the intranasal (i.n., 50 uL saline) route with 1×10E3 to 5×10E5 TCID50 of the Urbani strain of SARS-CoV at passage 2 in Vero cells. Various doses of ACF (e.g. range of 0.5-50 mg/kg) are given at the time of infection or at various times after infection. Animals are weighed and observed daily for sign of illness and mortality. Infected mice are sacrificed at various time intervals after inoculation.

Lung tissues are weighed and homogenized in phosphate-buffered saline (PBS) containing 10% fetal bovine serum using a TissueLyser (Qiagen). The resulting 10% tissue suspensions are clarified by centrifugation and subjected to virus titration with the standard infectivity assay using Vero E6 cells. The virus titer of individual samples is expressed as TCID50 per g of sample. The results demonstrate attenuated viral replication in mice treated with ACF.

Total RNA is isolated from tissues of infected mice at various time intervals after infection using an RNeasy Mini kit (Qiagen Sciences). Contaminating genomic DNA is removed with DNase I. RNA is amplified by quantitative real-time RT-PCR (qRT-PCR) analysis to assess the expression of SARS-CoV-specific subgenomic mRNA 1 and mRNA 5. The following primers and detection probes are used: for mRNA 5, forward, 5′-AGGTTTCCTATTCCTAGCCTGGATT, and reverse, 5′-AGAGCCAGAGGAAAACAAGCTTTAT, with 5′-ACCTGTTCCGATTAGAATAG as a detection probe; and for mRNA 1, forward, 5′-TCTGCGGATGCATCAACGT, and reverse, 5′-TGTAAGACGGGCTGCACTT, with 5′-CCGCAAACCCGTTTAAA as a detection probe. The selected primer set and Taq-Man probe for 18S rRNA (Applied Biosystems) are used as the endogenous control. To carry out the amplification reaction, 80 ng of RNA from each sampled time point is transferred to separate tubes for amplification of the target genes and endogenous control (18S rRNA), respectively, by using a TaqMan one-step RT-PCR master mix reagent kit (Applied Biosystems). The cycling parameters are as follows: reverse transcription at 48° C. for 30 min, AmpliTaq activation at 95° C. for 10 min, denaturation at 95° C. for 15 s, and annealing/extension at 60° C. for 1 min. A total of 40 cycles are performed on an ABI PRISM 7700 real-time thermocycler (Applied Biosystems) following the manufacturer's instructions. C0 values decrease with increasing time post-infection, indicating increasing viral titer. Treatment of mice with ACF decreases viral replication in the murine host, as reflected in higher C0 values post-infection.

Multiple tissues obtained from necropsy are fixed in 10% buffered formalin for 72 h, transferred to 70% ethanol, and later paraffin embedded. Histopathologic evaluation is performed on deparaffinized sections stained by routine hematoxylin-and-eosin staining. Histology shows reduced damage to lungs and other tissues following viral infection when ACF is given to mice.

Immunohistochemical (IHC) testing for SARS-CoV is applied using a colorimetric indirect immunoalkaline phosphatase method with a rabbit anti-SARS-CoV nucleocapsid protein antibody (Imgenex). Results indicate lower levels of SARS-CoV nucleocapsid protein, indicating lower viral replication in mice treated with ACF prior to or after infection.

Those skilled in the art will recognize, or be able to ascertain using no more than routine experimentation, many equivalents to the specific embodiments of the invention described herein. Such equivalents are intended to be encompassed by the following claims.

### REFERENCES

Belouzard S, Millet J K, Licitra, B N, and Whittaker, G R. Mechanisms of Coronavirus Cell Entry Mediated by the Viral Spike Protein. Viruses. 2012 June; 4(6): 1011-1033.
Campbell C M, Kahwash R. 2020. Will Complement Inhibition be the New Target in Treating COVID-19 Related Systemic Thrombosis? Circulation. https://doi.org/10.1161/CIRCULATIONAHA.120.047419.
Chang W C, White M R, Moyo P, McClear S, Thiel S, Hartshorn K L, Takahashi K. 2010. Lack of the pattern recognition molecule mannose binding lectin increases susceptibility to influenza A virus infection. BMC Immunol 11:64.
Chen R E, et al. 2021. Resistance of SARS-CoV-2 variants to neutralization by monoclonal and serum-derived polyclonal antibodies. Nature Medicine. https://doi.org/10.1038/s41591-021-01294-w.
Ehrnthaller C, et al. New Insights of an Old Defense System: Structure, Function, and Clinical Relevance of the Complement System. 2011, Mol. Med., 17: 317-329.
Gallagher T M, Buchmeier M J. Coronavirus spike proteins in viral entry and pathogenesis. Virology. 2001 Jan. 20; 279(2):371-4.
Gao T, Hu M. et al. 2020. Highly pathogenic coronavirus N protein aggravates lung injury by MASP-2-mediated complement over-activation. medRxiV. doi: https://doi.org/10.1101/2020.03.29.20041962.
Gralinski L E, et al. Complement Activation Contributes to Severe Acute Respiratory Syndrome Coronavirus Pathogenesis. mBio. 2018 Oct. 9; 9(5). pii: e01753-18.
Huang C, Wang Y, Li X, et al. 2020. Clinical features of patients infected with 2019 novel coronavirus in Wuhan, China. Lancet. 395: 497-506.
Imai Y, Kuba K, Ohto-Nakanishi T, Penninger J M. Angiotensin-converting enzyme 2 (ACE2) in disease pathogenesis. Circ J. 2010 March; 74(3):405-10.
Jia H, Pulmonary Angiotensin-Converting Enzyme 2 (ACE2) and Inflammatory Lung Disease. Shock. 2016 September; 46(3):239-48.
Jiang Y, et al. Blockade of the C5a-05aR axis alleviates lung damage in hDPP4-transgenic mice infected with MERS-CoV. Emerg Microbes Infect. 2018 Apr. 24; 7(1):77.
Khan A, et al. A pilot clinical trial of recombinant human angiotensin-converting enzyme 2 in acute respiratory distress syndrome. Crit Care. 2017; 21(1):234.
Krych-Goldberg M. Decay Accelerating Activity of Complement Receptor Type 1 (CD35). 1999, J. Bio. Chem., 274(44): 31160-31168
Krych-Goldberg M, et al. Structure-function relationships of complement receptor type 1. 2001, Immunological Reviews, 180: 112-122
Kuba K, et al. A crucial role of angiotensin converting enzyme 2 (ACE2) in SARS coronavirus-induced lung injury. Nat Med. 2005; 11(8):875-9.
Li F. Structure, Function, and Evolution of Coronavirus Spike Proteins. Annual review of virology. 2016 08/25; 3(1):237-61.
Liu J, Li S, Liu J, et al. Longitudinal characteristics of lymphocyte responses and cytokine profiles in the peripheral blood of SARS-CoV-2 infected patients. medRxiv 2020; published online February 22. DOI:10.1101/2020.02.16.20023671 (preprint).
Magro C, Mulvey J J, Berlin D, Nuovo G, Salvatore S, Harp J, Baxter-Stoltzfus A, Laurence J. 2020. Complement associated microvascular injury and thrombosis in the pathogenesis of severe COVID-19 infection: A report of five cases, Translational Research. doi: https://doi.org/10.1016/j.trs1.2020.04.007
Makrides S C. Therapeutic Inhibition of the Complement System. 1998, Pharmacological Reviews, 50 (1): 59-87
Mastellos D C, Ricklin D, Hajishengallis E, Hajishengallis G, Lambris J D. Complement therapeutics in inflammatory diseases: promising drug candidates for C3-targeted intervention. Mol Oral Microbiol. 2016 February; 31(1):3-17.
Millet J K, Whittaker G R. Murine Leukemia Virus (MLV)-based Coronavirus Spike-pseudotyped Particle Production and Infection. Bio Protoc. 2016 Dec. 5; 6(23).
Mollnes T E, et al. Strategies of therapeutic complement inhibition. 2006, Molecular Immunology, 43: 107-121
Patel V B, Zhong J C, Grant M B, Oudit G Y. Role of the ACE2/Angiotensin 1-7 Axis of the Renin-Angiotensin System in Heart Failure. Circ Res. 2016 Apr. 15; 118(8):1313-26.
Perry G J, et al. Phase I safety trial of soluble complement receptor type 1 (TP10) in acute myocardial infarction. 1998, J. Am. Coll. Cardiol., 31: 41 1A
Ricklin D, Lambris J D. New milestones ahead in complement-targeted therapy. Semin Immunol. 2016 June; 28(3):208-22.
Ricklin D, Barratt-Due A, Mollnes T E. Complement in clinical medicine: Clinical trials, case reports and therapy monitoring. Mol Immunol. 2017 September; 89:10-21.
Sarma V J, Huber-Lang M, Ward P A. 2006. Complement in lung disease. Autoimmunity 39:387-394.
Song W, Gui M, Wang X, Xiang Y. Cryo-E M structure of the SARS coronavirus spike glycoprotein in complex with its host cell receptor ACE2. PLoS Pathog. 2018 Aug. 13; 14(8)
Sun S, et al. 2013. Inhibition of complement activation alleviates acute lung injury induced by highly pathogenic avian influenza H5N1 virus infection. Am J Respir Cell Mol Biol 49:221-230.
Thielens N M, Tacnet-Delorme P, Arlaud G J. 2002. Interaction of C1q and mannan-binding lectin with viruses. Immunobiology 205:563-574.
Wagner E, et al. Therapeutic potential of complement modulation. 2010, Nat. Rev. Drug Discov., 9(1): 43-56.
Wang P, et al. 2021. Antibody Resistance of SARS-CoV-2 Variants B.1.351 and B.1.1.7. Nature. https://doi.org/10.1038/s41586-021-03398-2
Xu Z., Shi L., Wang Y. Pathologic findings of COVID-19 associated with acute respiratory distress syndrome. Lancet Respiratory Med. 2020; 8:420-422.
Zamora M R, et al. Complement inhibition attenuates human lung transplant reperfusion injury: a multicenter trial. 1999, Chest, 116: 46s
Zhang H, Penninger J M, Li Y, Zhong N, Slutsky A S. Angiotensin-converting enzyme 2 (ACE2) as a SARS-CoV-2 receptor: molecular mechanisms and potential therapeutic target. Intensive Care Med. 2020 Mar. 3.
Zhang H., Zhou P., Wei Y. 2020b. Histopathologic changes and SARS-Cov-2 immunostaining in the lung of a patient with COVID-19. Ann Intern Med. DOI: 10.7326/M20-0533
Zhou P, et al. A pneumonia outbreak associated with a new coronavirus of probable bat origin. Nature. 2020 March; 579(7798):270-273.
Zhu N, et al. A Novel Coronavirus from Patients with Pneumonia in China, 2019. N Engl J Med 382: 727-733.
Zimmerman J L, et al. Phase I trial of the recombinant soluble complement receptor 1 in acute lung injury and acute respiratory distress syndrome. 2000, Crit. Care. Med., 28(9): 3149-3154.
Zipfel P F, Wiech T, Rudnick R, Afonso S, Person F, Skerka C. Complement Inhibitors in Clinical Trials for Glomerular Diseases. Front Immunol. 2019 Sep. 27; 10:2166.
Zou Z, et al. Angiotensin-converting enzyme 2 protects from lethal avian influenza A H5N1 infections. Nat Commun. 2014; 5:3594.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

| SEQUENCES | |
|---|---|
| SEQ ID NO: 1 (nucleic acid) and SEQ ID NO: 2 (amino acid): Human ACE2 | |
| extracellular domain | |
| 1 | cagtccacca ttgaggaaca ggccaagaca tttttggaca agtttaacca |
| | Q S T I E E Q A K T F L D K F N |
| 51 | cgaagccgaa gacctgttct atcaaagttc acttgcttct tggaattata |
| | H E A E D L F Y Q S S L A S W N Y |
| 101 | acaccaatat tactgaagag aatgtccaaa acatgaataa tgctggggac |
| | N T N I T E E N V Q N M N N A G D |
| 151 | aaatggtctg cctttttaaa ggaacagtcc acacttgccc aaatgtatcc |
| | K W S A F L K E Q S T L A Q M Y |
| 201 | actacaagaa attcagaatc tcacagtcaa gcttcagctg caggctcttc |
| | P L Q E I Q N L T V K L Q L Q A L |
| 251 | agcaaaatgg gtcttcagtg ctctcagaag acaagagcaa acggttgaac |
| | Q Q N G S S V L S E D K S K P L N |
| 301 | acaattctaa atacaatgag caccatctac agtactggaa aagtttgtaa |
| | T I L N T M S T I Y S T G K V C |
| 351 | cccagataat ccacaagaat gcttattact tgaaccaggt ttgaatgaaa |
| | N P D N P Q E C L L L E P G L N E |
| 401 | taatggcaaa cagtttagac tacaatgaga ggctctgggc ttgggaaagc |
| | I M A N S L D Y N E R L W A W E S |
| 451 | tggagatctg aggtcggcaa gcagctgagg ccattatatg aagagtatgt |
| | W R S E V G K Q L R P L Y E E Y |
| 501 | ggtcttgaaa aatgagatgg caagagcaaa tcattatgag gactatgggg |
| | V V L K N E M A R A N H Y E D Y G |
| 551 | attattggag aggagactat gaagtaaatg gggtagatgg ctatgactac |
| | D Y W P G D Y E V N G V D G Y D Y |
| 601 | agccgcggcc agttgattga agatgtggaa catacctttg aagagattaa |
| | S R G Q L I E D V E H T F E E I |
| 651 | accattatat gaacatcttc atgcctatgt gagggcaaag ttgatgaatg |
| | K P L Y E H L H A Y V R A K L M N |
| 701 | cctatccttc ctatatcagt ccaattggat gcctccctgc tcatttgctt |
| | A Y P S Y I S P I G C L P A H L L |
| 751 | ggtgatatgt ggggtagatt ttggacaaat ctgtactctt tgacagttcc |
| | G D M W G P F W T N L Y S L T V |
| 801 | ctttggacag aaaccaaaca tagatgttac tgatgcaatg gtggaccagg |
| | P F G Q K P N I D V T D A M V D Q |
| 851 | cctgggatgc acagagaata ttcaaggagg ccgagaagtt ctttgtatct |
| | A W D A Q R I F K E A E K F F V S |
| 901 | gttggtcttc ctaatatgac tcaaggattc tgggaaaatt ccatgctaac |
| | V G L P N M T Q G F W E N S M L |
| 951 | ggacccagga aatgttcaga aagcagtctg ccatcccaca gcttgggacc |
| | T D P G N V Q K A V C H P T A W D |
| 1001 | tggggaaggg cgacttcagg atccttatgt gcacaaaggt gacaatggac |
| | L G K G D F R I L M C T K V T M D |
| 1051 | gacttcctga cagctcatca tgagatgggg catatccagt atgatatggc |
| | D F L T A H H E M G H I Q Y D M |
| 1101 | atatgctgca caaccttttc tgctaagaaa tggagctaat gaaggattcc |
| | A Y A A Q P F L L R N G A N E G F |
| 1151 | atgaagctgt tggggaaatc atgtcacttt ctgcagccac acctaagcat |
| | H E A V G E I M S L S A A T P K H |
| 1201 | ttaaaatcca ttggtcttct gtcacccgat tttcaagaag acaatgaaac |
| | L K S I G L L S P D F Q E D N E |
| 1251 | agaaataaac ttcctgctca aacaagcact cacgattgtt gggactctgc |
| | T E I N F L L K Q A L T I V G T L |
| 1301 | catttactta catgttagag aagtggaggt ggatggtctt taaaggggaa |
| | P F T Y M L E K W R W M V F K G E |
| 1351 | attcccaaag accagtggat gaaaaagtgg tgggagatga agcgagagat |
| | I P K D Q W M K K W W E M K P E |
| 1401 | agttggggtg gtggaacctg tgccccatga tgaaacatac tgtgaccccg |
| | I V G V V E P V P H D E T Y C D P |
| 1451 | catctctgtt ccatgtttct aatgattact cattcattcg atattacaca |
| | A S L F H V S N D Y S F I P Y Y T |
| 1501 | aggacccttt accaattcca gtttcaagaa gcactttgtc aagcagctaa |
| | R T L Y Q F Q F Q E A L C Q A A |
| 1551 | acatgaaggc cctctgcaca aatgtgacat ctcaaactct acagaagctg |
| | K H E G P L H K C D I S N S T E A |
| 1601 | gacagaaact gttcaatatg ctgaggcttg gaaaatcaga accctggacc |
| | G Q K L F N M L R L G K S E P W T |
| 1651 | ctagcattgg aaaatgttgt aggagcaaag aacatgaatg taaggccact |
| | L A L E N V V G A K N M N V R P |
| 1701 | gctcaactac tttgagccct tatttacctg gctgaaagac cagaacaaga |
| | L L N Y F E P L F T W L K D Q N K |
| 1751 | attcttttgt gggatggagt accgactgga gtccatatgc agaccaaagc |
| | N S F V G W S T D W S P Y A D Q S |
| 1801 | atcaaagtga ggataagcct aaaatcagct cttggagata aagcatatga |
| | I K V P I S L K S A L G D K A Y |
| 1851 | atggaacgac aatgaaatgt acctgttccg atcatctgtt gcatatgcta |
| | E W N D N E M Y L F R S S V A Y A |
| 1901 | tgaggcagta ctttttaaaa gtaaaaaatc agatgattct ttttggggag |
| | M R Q Y F L K V K N Q M I L F G E |
| 1951 | gaggatgtgc gagtggctaa tttgaaacca agaatctcct ttaatttctt |
| | E D V R V A N L K P R I S F N F |
| 2001 | tgtcactgca cctaaaaatg tgtctgatat cattcctaga actgaagttg |
| | F V T A P K N V S D I I P R T E V |
| 2051 | aaaaggccat caggatgtcc cggagccgta tcaatgatgc tttccgtctg |
| | E K A I R M S R S R I N D A F R L |
| 2101 | aatgacaaca gcctagagtt tctggggata cagccaacac ttggacctcc |
| | N D N S L E F L G I Q P T L G P |
| 2151 | taaccagccc cctgtttcc |
| | P N Q P P V S |
| SEQ ID NO: 3 (nucleic acid) and SEQ ID NO: 4 (amino acid): Human ACE2 | |
| extracellular variant | |
| 1 | cagtccacca ttgaggaaca ggccaagaca tttttggaca agtttaacca |
| | Q S T I E E Q A K T F L D K F N |
| 51 | cgaagccgaa gacctgttct atcaaagttc acttgcttct tggaattata |
| | H E A E D L F Y Q S S L A S W N Y |
| 101 | acaccaatat tactgaagag aatgtccaaa acatgaataa tgctggggac |
| | N T N I T E E N V Q N M N N A G D |
| 151 | aaatggtctg cctttttaaa ggaacagtcc acacttgccc aaatgtatcc |
| | K W S A F L K E Q S T L A Q M Y |
| 201 | actacaagaa attcagaatc tcacagtcaa gcttcagctg caggctcttc |
| | P L Q E I Q N L T V K L Q L Q A L |
| 251 | agcaaaatgg gtcttcagtg ctctcagaag acaagagcaa acggttgaac |
| | Q Q N G S S V L S E D K S K R L N |
| 301 | acaattctaa atacaatgag caccatctac agtactggaa aagtttgtaa |
| | T I L N T M S T I Y S T G K V C |
| 351 | cccagataat ccacaagaat gcttattact tgaaccaggt ttgaatgaaa |
| | N P D N P Q E C L L L E P G L N E |
| 401 | taatggcaaa cagtttagac tacaatgaga ggctctgggc ttgggaaagc |
| | I M A N S L D Y N E R L W A W E S |
| 451 | tggagatctg aggtcggcaa gcagctgagg ccattatatg aagagtatgt |
| | W P S E V G K Q L R P L Y E E Y |
| 501 | ggtcttgaaa aatgagatgg caagagcaaa tcattatgag gactatgggg |
| | V V L K N E M A R A N H Y E D Y G |
| 551 | attattggag aggagactat gaagtaaatg gggtagatgg ctatgactac |
| | D Y W R G D Y E V N G V D G Y D Y |
| 601 | agccgcggcc agttgattga agatgtggaa catacctttg aagagattaa |
| | S R G Q L I E D V E H T F E E I |
| 651 | accattatat gaacatcttc atgcctatgt gagggcaaag ttgatgaatg |
| | K P L Y E H L H A Y V R A K L M N |
| 701 | cctatccttc ctatatcagt ccaattggat gcctccctgc tcatttgctt |
| | A Y P S Y I S P I G C L P A H L L |
| 751 | ggtgatatgt ggggtagatt ttggacaaat ctgtactctt tgacagttcc |
| | G D M W G R F W T N L Y S L T V |
| 801 | ctttggacag aaaccaaaca tagatgttac tgatgcaatg gtggaccagg |
| | P F G Q K P N I D V T D A M V D Q |
| 851 | cctgggatgc acagagaata ttcaaggagg ccgagaagtt ctttgtatct |
| | A W D A Q P I F K E A E K F F V S |
| 901 | gttggtcttc ctaatatgac tcaaggattc tgggaaaatt ccatgctaac |
| | V G L P N M T Q G F W E N S M L |
| 951 | ggacccagga aatgttcaga aagcagtctg ccatcccaca gcttgggacc |
| | T D P G N V Q K A V C H P T A W D |
| 1001 | tggggaaggg cgacttcagg atccttatgt gcacaaaggt gacaatggac |
| | L G K G D F R I L M C T K V T M D |
| 1051 | gacttcctga cagctcatca tgagatgggg catatccagt atgatatggc |
| | D F L T A H H E M G H I Q Y D M |
| 1101 | atatgctgca caaccttttc tgctaagaaa tggagctaat gaaggattcc |
| | A Y A A Q P F L L P N G A N E G F |
| 1151 | atgaagctgt tggggaaatc atgtcacttt ctgcagccac acctaagcat |
| | H E A V G E I M S L S A A T P K H |
| 1201 | ttaaaatcca ttggtcttct gtcacccgat tttcaagaag acaatgaaac |
| | L K S I G L L S P D F Q E D N E |
| 1251 | agaaataaac ttcctgctca aacaagcact cacgattgtt gggactctgc |
| | T E I N F L L K Q A L T I V G T L |
| 1301 | catttactta catgttagag aagtggaggt ggatggtctt taaaggggaa |
| | P F T Y M L E K W R W M V F K G E |
| 1351 | attcccaaag accagtggat gaaaaagtgg tgggagatga agcgagagat |
| | I P K D Q W M K K W W E M K R E |
| 1401 | agttggggtg gtggaacctg tgccccatga tgaaacatac tgtgaccccg |
| | I V G V V E P V P H D E T Y C D P |
| 1451 | catctctgtt ccatgtttct aatgattact cattcattcg atattacaca |
| | A S L F H V S N D Y S F I P Y Y T |
| 1501 | aggacccttt accaattcca gtttcaagaa gcactttgtc aagcagctaa |
| | R T L Y Q F Q F Q E A L C Q A A |
| 1551 | acatgaaggc cctctgcaca aatgtgacat ctcaaactct acagaagctg |
| | K H E G P L H K C D I S N S T E A |
| 1601 | gacagaaact gttcaatatg ctgaggcttg gaaaatcaga accctggacc |
| | G Q K L F N M L R L G K S E P W T |
| 1651 | ctagcattgg aaaatgttgt aggagcaaag aacatgaatg taaggccact |
| | L A L E N V V G A K N M N V R P |
| 1701 | gctcaactac tttgagccct tatttacctg gctgaaagac cagaacaaga |
| | L L N Y F E P L F T W L K D Q N K |
| 1751 | attcttttgt gggatggagt accgactgga gtccatatgc agac |
| | N S F V G W S T D W S P Y A D |
| SEQ ID NO: 5 (nucleic acid) and SEQ ID NO: 6 (amino acid): Human ACE2 | |
| extracellular variant | |
| 1 | cagtccacca ttgaggaaca ggccaagaca tttttggaca agtttaacca |
| | Q S T I E E Q A K T F L D K F N |
| 51 | cgaagccgaa gacctgttct atcaaagttc acttgcttct tggaattata |
| | H E A E D L F Y Q S S L A S W N Y |
| 101 | acaccaatat tactgaagag aatgtccaaa acatgaataa tgctggggac |
| | N T N I T E E N V Q N M N N A G D |
| 151 | aaatggtctg cctttttaaa ggaacagtcc acacttgccc aaatgtatcc |
| | K W S A F L K E Q S T L A Q M Y |
| 201 | actacaagaa attcagaatc tcacagtcaa gcttcagctg caggctcttc |
| | P L Q E I Q N L T V K L Q L Q A L |
| 251 | agcaaaatgg gtcttcagtg ctctcagaag acaagagcaa acggttgaac |
| | Q Q N G S S V L S E D K S K R L N |
| 301 | acaattctaa atacaatgag caccatctac agtactggaa aagtttgtaa |
| | T I L N T M S T I Y S T G K V C |
| 351 | cccagataat ccacaagaat gcttattact tgaaccaggt ttgaatgaaa |
| | N P D N P Q E C L L L E P G L N E |
| 401 | taatggcaaa cagtttagac tacaatgaga ggctctgggc ttgggaaagc |
| | I M A N S L D Y N E R L W A W E S |
| 451 | tggagatctg aggtcggcaa gcagctgagg ccattatatg aagagtatgt |
| | W R S E V G K Q L R P L Y E E Y |
| 501 | ggtcttgaaa aatgagatgg caagagcaaa tcattatgag gactatgggg |
| | V V L K N E M A R A N H Y E D Y G |
| 551 | attattggag aggagactat gaagtaaatg gggtagatgg ctatgactac |
| | D Y W R G D Y E V N G V D G Y D Y |
| 601 | agccgcggcc agttgattga agatgtggaa catacctttg aagagattaa |
| | S R G Q L I E D V E H T F E E I |
| 651 | accattatat gaacatcttc atgcctatgt gagggcaaag ttgatgaatg |
| | K P L Y E H L H A Y V R A K L M N |
| 701 | cctatccttc ctatatcagt ccaattggat gcctccctgc tcatttgctt |
| | A Y P S Y I S P I G C L P A H L L |
| 751 | ggtgatatgt ggggtagatt ttggacaaat ctgtactctt tgacagttcc |
| | G D M W G R F W T N L Y S L T V |
| 801 | ctttggacag aaaccaaaca tagatgttac tgatgcaatg gtggaccagg |
| | P F G Q K P N I D V T D A M V D Q |
| 851 | cctgggatgc acagagaata ttcaaggagg ccgagaagtt ctttgtatct |
| | A W D A Q R I F K E A E K F F V S |
| 901 | gttggtcttc ctaatatgac tcaaggattc tgggaaaatt ccatgctaac |
| | V G L P N M T Q G F W E N S M L |
| 951 | ggacccagga aatgttcaga aagcagtctg ccatcccaca gcttgggacc |
| | T D P G N V Q K A V C H P T A W D |
| 1001 | tggggaaggg cgacttcagg atccttatgt gcacaaaggt gacaatggac |
| | L G K G D F R I L M C T K V T M D |
| 1051 | gacttcctga cagctcatca tgagatgggg catatccagt atgatatggc |
| | D F L T A H H E M G H I Q Y D M |
| 1101 | atatgctgca caaccttttc tgctaagaaa tggagctaat gaaggattcc |
| | A Y A A Q P F L L R N G A N E G F |
| 1151 | atgaagctgt tggggaaatc atgtcacttt ctgcagccac acctaagcat |
| | H E A V G E I M S L S A A T P K H |
| 1201 | ttaaaatcca ttggtcttct gtcacccgat tttcaagaag acaatgaaac |
| | L K S I G L L S P D F Q E D N E |
| 1251 | agaaataaac ttcctgctca aacaagcact cacgattgtt gggactctgc |
| | T E I N F L L K Q A L T I V G T L |
| 1301 | catttactta catgttagag aagtggaggt ggatggtctt taaaggggaa |
| | P F T Y M L E K W R W M V F K G E |
| 1351 | attcccaaag accagtggat gaaaaagtgg tgggagatga agcgagagat |
| | I P K D Q W M K K W W E M K R E |
| 1401 | agttggggtg gtggaacctg tgccccatga tgaaacatac tgtgaccccg |
| | I V G V V E P V P H D E T Y C D P |
| 1451 | catctctgtt ccatgtttct aatgattact cattcattcg atattacaca |
| | A S L F H V S N D Y S F I R Y Y T |
| 1501 | aggacccttt accaattcca gtttcaagaa gcactttgtc aagcagctaa |
| | R T L Y Q F Q F Q E A L C Q A A |
| 1551 | acatgaaggc cctctgcaca aatgtgacat ctcaaactct acagaagctg |
| | K H E G P L H K C D I S N S T E A |
| 1601 | gacagaaact gttcaatatg ctgaggcttg gaaaatcaga accctggacc |
| | G Q K L F N M L R L G K S E P W T |
| 1651 | ctagcattgg aaaatgttgt aggagcaaag aacatgaatg taaggccact |
| | L A L E N V V G A K N M N V R P |
| 1701 | gctcaactac tttgagccct tatttacctg gctgaaagac cagaacaaga |
| | L L N Y F E P L F T W L K D Q N K |
| 1751 | attcttttgt gggatggagt accgactgga gtccatatgc agac |
| | N S F V G W S T D W S P Y A D |
| SEQ ID NO: 7 (nucleic acid) and SEQ ID NO: 8 (amino acid): Human ACE2 | |
| extracellular variant | |
| 1 | cagtccacca ttgaggaaca ggccaagaca tttttggaca agtttaacca |
| | Q S T I E E Q A K T F L D K F N |
| 51 | cgaagccgaa gacctgttct atcaaagttc acttgcttct tggaattata |
| | H E A E D L F Y Q S S L A S W N Y |
| 101 | acaccaatat tactgaagag aatgtccaaa acatgaataa tgctggggac |
| | N T N I T E E N V Q N M N N A G D |
| 151 | aaatggtctg cctttttaaa ggaacagtcc acacttgccc aaatgtatcc |
| | K W S A F L K E Q S T L A Q M Y |
| 201 | actacaagaa attcagaatc tcacagtcaa gcttcagctg caggctcttc |
| | P L Q E I Q N L T V K L Q L Q A L |
| 251 | agcaaaatgg gtcttcagtg ctctcagaag acaagagcaa acggttgaac |
| | Q Q N G S S V L S E D K S K R L N |
| 301 | acaattctaa atacaatgag caccatctac agtactggaa aagtttgtaa |
| | T I L N T M S T I Y S T G K V C |
| 351 | cccagataat ccacaagaat gcttattact tgaaccaggt ttgaatgaaa |
| | N P D N P Q E C L L L E P G L N E |
| 401 | taatggcaaa cagtttagac tacaatgaga ggctctgggc ttgggaaagc |
| | I M A N S L D Y N E R L W A W E S |
| 451 | tggagatctg aggtcggcaa gcagctgagg ccattatatg aagagtatgt |
| | W R S E V G K Q L R P L Y E E Y |
| 501 | ggtcttgaaa aatgagatgg caagagcaaa tcattatgag gactatgggg |
| | V V L K N E M A R A N H Y E D Y G |
| 551 | attattggag aggagactat gaagtaaatg gggtagatgg ctatgactac |
| | D Y W R G D Y E V N G V D G Y D Y |
| 601 | agccgcggcc agttgattga agatgtggaa catacctttg aagagattaa |
| | S P G Q L I E D V E H T F E E I |
| 651 | accattatat gaacatcttc atgcctatgt gagggcaaag ttgatgaatg |
| | K P L Y E H L H A Y V R A K L M N |
| 701 | cctatccttc ctatatcagt ccaattggat gcctccctgc tcatttgctt |
| | A Y P S Y I S P I G C L P A H L L |
| 751 | ggtgatatgt ggggtagatt ttggacaaat ctgtactctt tgacagttcc |
| | G D M W G P F W T N L Y S L T V |
| 801 | ctttggacag aaaccaaaca tagatgttac tgatgcaatg gtggaccagg |
| | P F G Q K P N I D V T D A M V D Q |
| 851 | cctgggatgc acagagaata ttcaaggagg ccgagaagtt ctttgtatct |
| | A W D A Q R I F K E A E K F F V S |
| 901 | gttggtcttc ctaatatgac tcaaggattc tgggaaaatt ccatgctaac |
| | V G L P N M T Q G F W E N S M L |
| 951 | ggacccagga aatgttcaga aagcagtctg ccatcccaca gcttgggacc |
| | T D P G N V Q K A V C H P T A W D |
| 1001 | tggggaaggg cgacttcagg atccttatgt gcacaaaggt gacaatggac |
| | L G K G D F R I L M C T K V T M D |
| 1051 | gacttcctga cagctcataa cgagatgggg aatatccagt atgatatggc |
| | D F L T A H N E M G N I Q Y D M |
| 1101 | atatgctgca caaccttttc tgctaagaaa tggagctaat gaaggattcc |
| | A Y A A Q P F L L R N G A N E G F |
| 1151 | atgaagctgt tggggaaatc atgtcacttt ctgcagccac acctaagcat |
| | H E A V G E I M S L S A A T P K H |
| 1201 | ttaaaatcca ttggtcttct gtcacccgat tttcaagaag acaatgaaac |
| | L K S I G L L S P D F Q E D N E |
| 1251 | agaaataaac ttcctgctca aacaagcact cacgattgtt gggactctgc |
| | T E I N F L L K Q A L T I V G T L |
| 1301 | catttactta catgttagag aagtggaggt ggatggtctt taaaggggaa |
| | P F T Y M L E K W R W M V F K G E |
| 1351 | attcccaaag accagtggat gaaaaagtgg tgggagatga agcgagagat |
| | I P K D Q W M K K W W E M K R E |
| 1401 | agttggggtg gtggaacctg tgccccatga tgaaacatac tgtgaccccg |
| | I V G V V E P V P H D E T Y C D P |
| 1451 | catctctgtt ccatgtttct aatgattact cattcattcg atattacaca |
| | A S L F H V S N D Y S F I R Y Y T |
| 1501 | aggacccttt accaattcca gtttcaagaa gcactttgtc aagcagctaa |
| | P T L Y Q F Q F Q E A L C Q A A |
| 1551 | acatgaaggc cctctgcaca aatgtgacat ctcaaactct acagaagctg |
| | K H E G P L H K C D I S N S T E A |
| 1601 | gacagaaact gttcaatatg ctgaggcttg gaaaatcaga accctggacc |
| | G Q K L F N M L R L G K S E P W T |
| 1651 | ctagcattgg aaaatgttgt aggagcaaag aacatgaatg taaggccact |
| | L A L E N V V G A K N M N V R P |
| 1701 | gctcaactac tttgagccct tatttacctg gctgaaagac cagaacaaga |
| | L L N Y F E P L F T W L K D Q N K |
| 1751 | attcttttgt gggatggagt accgactgga gtccatatgc agac |
| | N S F V G W S T D W S P Y A D |
| SEQ ID NO: 9 (nucleic acid) and SEQ ID NO: 10 (amino acid): Human | |
| CR1 SCR1-3 | |
| 1 | caatgcaatg ccccagaatg gcttccattt gccaggccta ccaacctaac tgatgaattt |
| | Q C N A P E W L P F A R P T N L T D E F |
| 61 | gagtttccca ttgggacata tctgaactat gaatgccgcc ctggttattc cggaagaccg |
| | E F P I G T Y L N Y E C R P G Y S G P P |
| 121 | ttttctatca tctgcctaaa aaactcagtc tggactggtg ctaaggacag gtgcagacgt |
| | F S I I C L K N S V W T G A K D R C R R |
| 181 | aaatcatgtc gtaatcctcc agatcctgtg aatggcatgg tgcatgtgat caaaggcatc |
| | K S C R N P P D P V N G M V H V I K G I |
| 241 | cagttcggat cccaaattaa atattcttgt actaaaggat accgactcat tggttcctcg |
| | Q F G S Q I K Y S C T K G Y R L I G S S |
| 301 | tctgccacat gcatcatctc aggtgatact gtcatttggg ataatgaaac acctatttgt |
| | S A T C I I S G D T V I W D N E T P I C |
| 361 | gacagaattc cttgtgggct accccccacc atcaccaatg gagatttcat tagcaccaac |
| | D R I P C G L P P T I T N G D F I S T N |
| 421 | agagagaatt ttcactatgg atcagtggtg acctaccgct gcaatcctgg aagcggaggg |
| | R E N F H Y G S V V T Y R C N P G S G G |
| 481 | agaaaggtgt ttgagcttgt gggtgagccc tccatatact gcaccagcaa tgacgatcaa |
| | R K V F E L V G E P S I Y C T S N D D Q |
| 541 | gtgggcatct ggagcggccc cgcccctcag tgcatt |
| | V G I W S G P A P Q C I |
| SEQ ID NO: 11 (nucleic acid) and SEQ ID NO: 12 (amino acid): Human CR1 SCR1- | |
| 3_N29K/S37Y/G79N/D109N | |
| 1 | caatgcaatg ccccagaatg gcttccattt gccaggccta ccaacctaac tgatgaattt |
| | Q C N A P E W L P F A R P T N L T D E F |
| 61 | gagtttccca ttgggacata tctgaaatat gaatgccgcc ctggttatta cggaagaccg |
| | E F P I G T Y L K Y E C R P G Y Y G R P |
| 121 | ttttctatca tctgcctaaa aaactcagtc tggactggtg ctaaggacag gtgcagacgt |
| | F S I I C L K N S V W T G A K D R C R R |
| 181 | aaatcatgtc gtaatcctcc agatcctgtg aatggcatgg tgcatgtgat caaagacatc |
| | K S C P N R P D P V N G M V H V I K D I |
| 241 | cagttcggat cccaaattaa atattcttgt actaaaggat accgactcat tggttcctcg |
| | Q F G S Q I K Y S C T K G Y R L I G S S |
| 301 | tctgccacat gcatcatctc aggtaatact gtcatttggg ataatgaaac acctatttgt |
| | S A T C I I S G N T V I W D N E T P I C |
| 361 | gacagaattc cttgtgggct accccccacc atcaccaatg gagatttcat tagcaccaac |
| | D R I P C G L P P T I T N G D F I S T N |
| 421 | agagagaatt ttcactatgg atcagtggtg acctaccgct gcaatcctgg aagcggaggg |
| | R E N F H Y G S V V T Y R C N P G S G G |
| 481 | agaaaggtgt ttgagcttgt gggtgagccc tccatatact gcaccagcaa tgacgatcaa |
| | R K V F E L V G E P S I Y C T S N D D Q |
| 541 | gtgggcatct ggagcggccc cgcccctcag tgcatt |
| | V G I W S G P A P Q C I |
| SEQ ID NO: 13 (nucleic acid) and SEQ ID NO: 14 (amino acid): DAF SCR2-4 | |
| 1 | cgtagctgcg aggtgccaac aaggctaaat tctgcatccc tcaaacagcc ttatatcact |
| | R S C E V P T R L N S A S L K Q P Y I T |
| 61 | cagaattatt ttccagtcgg tactgttgtg gaatatgagt gccgtccagg ttacagaaga |
| | Q N Y F P V G T V V E Y E C R P G Y P R |
| 121 | gaaccttctc tatcaccaaa actaacttgc cttcagaatt taaaatggtc cacagcagtc |
| | E P S L S P K L T C L Q N L K W S T A V |
| 181 | gaattttgta aaaagaaatc atgccctaat ccgggagaaa tacgaaatgg tcagattgat |
| | E F C K K K S C P N P G E I R N G Q I D |
| 241 | gtaccaggtg gcatattatt tggtgcaacc atctccttct catgtaacac agggtacaaa |
| | V P G G I L F G A T I S F S C N T G Y K |
| 301 | ttatttggct cgacttctag tttttgtctt atttcaggca gctctgtcca gtggagtgac |
| | L F G S T S S F C L I S G S S V Q W S D |
| 361 | ccgttgccag agtgcagaga aatttattgt ccagcaccac cacaaattga caatggaata |
| | P L P E C R E I Y C P A P P Q I D N G I |
| 421 | attcaagggg aacgtgacca ttatggatat agacagtctg taacgtatgc atgtaataaa |
| | I Q G E R D H Y G Y R Q S V T Y A C N K |
| 481 | ggattcacca tgattggaga gcactctatt tattgtactg tgaataatga tgaaggagag |
| | G F T M I G E H S I Y C T V N N D E G E |
| 541 | tggagtggcc caccacctga atgcaga |
| | W S G P P P E C R |
| SEQ ID NO: 15 (nucleic acid) and SEQ ID NO: 16 (amino acid): MCP SCR2-4 | |
| 1 | agagaaacat gtccatatat acgggatcct ttaaatggcc aagcagtccc tgcaaatggg |
| | R E T C P Y I R D P L N G Q A V P A N G |
| 61 | acttacgagt ttggttatca gatgcacttt atttgtaatg agggttatta cttaattggt |
| | T Y E F G Y Q M H F I C N E G Y Y L I G |
| 121 | gaagaaattc tatattgtga acttaaagga tcagtagcaa tttggagcgg taagccccca |
| | E E I L Y C E L K G S V A I W S G K P P |
| 181 | atatgtgaaa aggttttgtg tacaccacct ccaaaaataa aaaatggaaa acacaccttt |
| | I C E K V L C T P P P K I K N G K H T F |
| 241 | agtgaagtag aagtatttga gtatcttgat gcagtaactt atagttgtga tcctgcacct |
| | S E V E V F E Y L D A V T Y S C D P A P |
| 301 | ggaccagatc cattttcact tattggagag agcacgattt attgtggtga caattcagtg |
| | G P D P F S L I G E S T I Y C G D N S V |
| 361 | tggagtcgtg ctgctccaga gtgtaaagtg gtcaaatgtc gatttccagt agtcgaaaat |
| | W S R A A P E C K V V K C R F P V V E N |
| 421 | ggaaaacaga tatcaggatt tggaaaaaaa ttttactaca aagcaacagt tatgtttgaa |
| | G K Q I S G F G K K F Y Y K A T V M F E |
| 481 | tgcgataagg gtttttacct cgatggcagc gacacaattg tctgtgacag taacagtact |
| | C D K G F Y L D G S D T I V C D S N S T |
| 541 | tgggatcccc cagttccaaa gtgtctt |
| | W D P P V P K C L |
| SEQ ID NO: 17 (nucleic acid) and SEQ ID NO: 18 (amino acid): Factor H SCR1-4 | |
| 1 | gaagattgca atgaacttcc tccaagaaga aatacagaaa ttctgacagg ttcctggtct |
| | E D C N E L P P R R N T E I L T G S W S |
| 61 | gaccaaacat atccagaagg cacccaggct atctataaat gccgccctgg atatagatct |
| | D Q T Y P E G T Q A I Y K C R P G Y R S |
| 121 | cttggaaatg taataatggt atgcaggaag ggagaatggg ttgctcttaa tccattaagg |
| | L G N V I M V C R K G E W V A L N P L R |
| 181 | aaatgtcaga aaaggccctg tggacatcct ggagatactc cttttggtac ttttaccctt |
| | K C Q K R P C G H P G D T P F G T F T L |
| 241 | acaggaggaa atgtgtttga atatggtgta aaagctgtgt atacatgtaa tgaggggtat |
| | T G G N V F E Y G V K A V Y T C N E G Y |
| 301 | caattgctag gtgagattaa ttaccgtgaa tgtgacacag atggatggac caatgatatt |
| | Q L L G E I N Y R E C D T D G W T N D I |
| 361 | cctatatgtg aagttgtgaa gtgtttacca gtgacagcac cagagaatgg aaaaattgtc |
| | P I C E V V K C L P V T A P E N G K I V |
| 421 | agtagtgcaa tggaaccaga tcgggaatac cattttggac aagcagtacg gtttgtatgt |
| | S S A M E P D R E Y H F G Q A V R F V C |
| 481 | aactcaggct acaagattga aggagatgaa gaaatgcatt gttcagacga tggtttttgg |
| | N S G Y K I E G D E E M H C S D D G F W |
| 541 | agtaaagaga aaccaaagtg tgtggaaatt tcatgcaaat ccccagatgt tataaatgga |
| | S K E K P K C V E I S C K S P D V I N G |
| 601 | tctcctatat ctcagaagat tatttataag gagaatgaac gatttcaata taaatgtaac |
| | S P I S Q K I I Y K E N E R F Q Y K C N |
| 661 | atgggttatg aatacagtga aagaggagat gctgtatgca ctgaatctgg atggcgtccg |
| | M G Y E Y S E R G D A V C T E S G W R P |
| 721 | ttgccttcat gtgaa |
| | L P S C E |
| SEQ ID NO: 19 (nucleic acid) and SEQ ID NO: 20 (amino acid): C4BPA SCR1-3 | |
| 1 | aattgtggtc ctccacccac tttatcattt gctgccccga tggatattac gttgactgag |
| | N C G P P P T L S F A A P M D I T L T E |
| 61 | acacgcttca aaactggaac tactctgaaa tacacctgcc tccctggcta cgtcagatcc |
| | T R F K T G T T L K Y T C L P G Y V R S |
| 121 | cattcaactc agacgcttac ctgtaattct gatggcgaat gggtgtataa caccttctgt |
| | H S T Q T L T C N S D G E W V Y N T F C |
| 181 | atctacaaac gatgcagaca cccaggagag ttacgtaatg ggcaagtaga gattaagaca |
| | I Y K R C R H P G E L R N G Q V E I K T |
| 241 | gatttatctt ttggatcaca aatagaattc agctgttcag aaggattttt cttaattggc |
| | D L S F G S Q I E F S C S E G F F L I G |
| 301 | tcaaccacta gtcgttgtga agtccaagat agaggagttg gctggagtca tcctctccca |
| | S T T S R C E V Q D R G V G W S H P L P |
| 361 | caatgtgaaa ttgtcaagtg taagcctcct ccagacatca ggaatggaag gcacagcggt |
| | Q C E I V K C K P P P D I R N G R H S G |
| 421 | gaagaaaatt tctacgcata cggcttttct gtcacctaca gctgtgaccc ccgcttctca |
| | E E N F Y A Y G F S V T Y S C D P R F S |
| 481 | ctcttgggcc atgcctccat ttcttgcact gtggagaatg aaacaatagg tgtttggaga |
| | L L G H A S I S C T V E N E T I G V W R |
| 541 | ccaagccctc ctacctgtga a |
| | P S P P T C E |

What is claimed is:

1. A fusion protein comprising, an ACE2 domain and a second polypeptide domain.

2. The fusion protein of claim 1, wherein the ACE2 domain is a full length extracellular domain of human ACE2, a deletion variant of human ACE2 or a human ACE2 variant with higher affinity for a coronavirus spike protein, and the second polypeptide domain is a complement inhibiting domain.

3. The fusion protein of claim 2, wherein the ACE2 domain is a SEQ ID NO: 2, SEQ ID NO: 4, SEQ ID NO: 6, or SEQ ID NO: 8.

4. The fusion protein of claim 2, wherein the complement inhibiting domain is a portion of a CR1, a DAF, a MCP, a Factor H, or a C4BP.

5. The fusion protein of claim 2, wherein the complement inhibiting domain is a SEQ ID NO: 10, SEQ ID NO: 12, SEQ ID NO: 14, SEQ ID NO: 16, SEQ ID NO: 18, or SEQ ID NO: 20.

6. The fusion protein of claim 2, wherein the complement inhibiting domain is an antibody fragment, or a scFv, or a variable region (VH or VK) from an antibody against a Factor B, a Factor D, a Factor P, a C3, or a C5.

7. The fusion protein of claim 2, wherein the complement inhibiting domain is a peptide inhibitor or an oligonucleotide inhibitor of a Factor B, a Factor D, a Factor P, a C3, or a C5.

8. The fusion protein of claim 2, wherein the ACE2 domain comprises an amino acid sequence of SEQ ID NO: 2, SEQ ID NO:4, SEQ ID NO:6 or SEQ ID NO:8 with a double amino acid substitution of K26R/L79W, T27R/L79W, F28W/L79W, D30E/L79W, or Q42E/L79W.

9. The fusion protein of claim 2, wherein the ACE2 domain comprises an amino acid sequence of SEQ ID NO: 2, SEQ ID NO:4, SEQ ID NO:6 or SEQ ID NO:8 with a triple amino acid substitution of K26R/D30E/L79W, D30E/L79W/N330Q, D30E/L79W/N330Y, or D30E/Q42K/L79W.

10. The fusion protein of claim 2, wherein the ACE2 domain comprises an amino acid sequence of SEQ ID NO: 2, SEQ ID NO:4, SEQ ID NO:6 or SEQ ID NO:8 with a quadruple amino acid substitution of K26R/F28W/D30E/L79W, D30E/Q42K/L79W/N330Q, D30E/Q42K/L79W/N330Y, or K31F/N33D/H34S/E35Q.

11. The fusion protein of claim 2, wherein the ACE2 domain comprises an amino acid sequence of SEQ ID NO: 2, SEQ ID NO:4, SEQ ID NO:6 or SEQ ID NO:8 with a quintuple amino acid substitution of K26R/T27R/F28W/D30E/L79W, T27R/D30E/Q42K/L79W/N330Y, T27Y/D30E/Q42K/L79W/N330Y, or D30E/H34V/Q42K/L79W/N330Y.

12. The fusion protein of claim 2, wherein the ACE2 domain comprises an amino acid sequence of SEQ ID NO: 2, SEQ ID NO:4, SEQ ID NO:6 or SEQ ID NO:8 with a septuple amino acid substitution D30E/K31F/H34I/E35Q/Q42K/L79W/N330Y.

13. The fusion protein of claim 2, further comprising a third polypeptide domain that is a half-life prolonging domain.

14. The fusion protein of claim 2, wherein the third domain is an immunoglobulin Fc region, wherein the Fc region is a wild-type or a variant of any human immunoglobulin isotype, subclass, or allotype, or a human albumin or any peptide that binds to a human albumin, or a portion of a human collagen.

15. The fusion protein of claim 1, wherein the ACE2 domain comprises an amino acid sequence of SEQ ID NO: 2, SEQ ID NO:4, SEQ ID NO:6 or SEQ ID NO:8 with a double amino acid substitution of K26R/L79W, T27R/L79W, F28W/L79W, D30E/L79W, or Q42E/L79W.

16. The fusion protein of claim 1, wherein the ACE2 domain comprises an amino acid sequence of SEQ ID NO: 2, SEQ ID NO:4, SEQ ID NO:6 or SEQ ID NO:8 with a triple amino acid substitution of K26R/D30E/L79W, D30E/L79W/N330Q, D30E/L79W/N330Y, or D30E/Q42K/L79W.

17. The fusion protein of claim 1, wherein the ACE2 domain comprises an amino acid sequence of SEQ ID NO: 2, SEQ ID NO:4, SEQ ID NO:6 or SEQ ID NO:8 with a quadruple amino acid substitution of K26R/F28W/D30E/L79W, D30E/Q42K/L79W/N330Q, D30E/Q42K/L79W/N330Y, or K31F/N33D/H34S/E35Q.

18. The fusion protein of claim 1, wherein the ACE2 domain comprises an amino acid sequence of SEQ ID NO: 2, SEQ ID NO:4, SEQ ID NO:6 or SEQ ID NO:8 with a quintuple amino acid substitution of K26R/T27R/F28W/D30E/L79W, T27R/D30E/Q42K/L79W/N330Y, T27Y/D30E/Q42K/L79W/N330Y, or D30E/H34V/Q42K/L79W/N330Y.

19. The fusion protein of claim 1, wherein the ACE2 domain comprises an amino acid sequence of SEQ ID NO: 2, SEQ ID NO:4, SEQ ID NO:6 or SEQ ID NO:8 with a septuple amino acid substitution D30E/K31F/H34I/E35Q/Q42K/L79W/N330Y.

20. The fusion protein of claim 1, wherein the second domain is an immunoglobulin Fc region, wherein the Fc region is a wild-type or a variant of any human immunoglobulin isotype, subclass, or allotype, or a human albumin or any peptide that binds to a human albumin, or a portion of a human collagen.
